(19) United States
(12) Patent Application Publication
(10) Pub. No.: US 2025/0049818 A1
(43) Pub. Date: Feb. 13, 2025

(54) PRENATAL SUPPLEMENT COMPOSITIONS FOR INCREASING BREASTMILK MICRONUTRIENT LEVELS

(71) Applicant: SOCIÉTÉ DES PRODUITS NESTLÉ S.A., Vevey (CH)

(72) Inventors: WAYNE S. CUTFIELD, Auckland (NZ); KEITH MALCOLM GODFREY, Ashurst Hampshire (GB); SHIAO-YNG CHAN, Singapore (SG); SOO MIN HAN, Auckland (NZ); MARK H. VICKERS, Auckland (NZ); JOSE G. B. DERRAIK, Auckland (NZ); IRMA SILVA ZOLEZZI, Jongny (CH); SAGAR THAKKAR, La Tour de Peilz (CH); FANG HUANG, Beijing (CN); SURABHI DEVARAJ, Fort Gardens (SG)

(21) Appl. No.: 18/717,836

(22) PCT Filed: Dec. 7, 2022
(86) PCT No.: PCT/EP2022/084813

§ 371 Date: Jun. 7, 2024

Related U.S. Application Data
(60) Provisional application No. 63/287,782, filed on Dec. 9, 2021.
(60) Provisional application No. 63/353,991, filed on Jun. 21, 2022.

(51) Int. Cl. A61K 31/593; A61K 31/015; A61K 31/047; A61K 31/4415; A61K 31/519; A61K 31/525; A61K 31/714; A61K 33/06; A61K 33/18; A61K 33/26; A61K 33/30; A61K 35/00; A61K 35/745; A61K 35/747

(52) U.S. Cl. CPC A61K 31/593; A61K 31/015; A61K 31/047; A61K 31/4415; A61K 31/519; A61K 31/525; A61K 31/714; A61K 33/06; A61K 33/18; A61K 33/26; A61K 33/30; A61K 35/745; A61K 35/747; A61K 2035/115

(57) ABSTRACT

A method increases a level of at least one micronutrient, such as vitamin D and/or zinc, in a subject's breastmilk. The method includes administering to the subject a nutritional composition containing at least one of vitamin D and zinc. The nutritional composition can include one or more of vitamins B₂, B₆ or B₁₂.

### BACKGROUND

The present disclosure generally relates to compositions and methods for increasing breastmilk micronutrient levels.

Supplementing women from before pregnancy can impact the health of their babies later in life (e.g., folic acid supplementation to decrease risk of fetal/infant neural tubes defects). Clinical evidence of the impact of preconception and pregnancy supplementation (e.g., a combination of preconception and pregnancy supplementation) with nutrient mixes on maternal nutritional status, human milk composition and different infant outcomes is either limited or non-existing.

Multi-micronutrient supplementations are common in pregnancy but their impact on human milk levels is not known. Limited evidence exists on the role of supplementation during lactation to improve the levels, which could be critical to ensure a healthy start in life for the newborn breastfed infant. For example, Zinc and vitamin D are two important micronutrients to human in general.

While there were no studies found that exclusively supplemented and investigated the effects of zinc supplementation during pregnancy on human milk zinc levels, one study from Indonesia measured the effect of combined supplementation of beta-carotene and/or zinc along with iron (control group) and folic acid (control group). Briefly, 170 pregnant women were supplemented during pregnancy only (recruitment <20 weeks of gestation) with a daily dose of beta-carotene (4.5 mg), zinc (30 mg), or both or placebo: iron (30 mg) plus folic acid (0.4 mg). Their micronutrient status was assessed at 1 and 6 months postpartum and human milk samples were collected at the same timepoints. Human milk concentrations of retinol and zinc decreased significantly between the first and sixth month of lactation in all groups combined. The zinc content of human milk at 6 months postpartum was not affected by either zinc or beta-carotene supplementation. Zinc concentrations did not significantly change in the supplemented groups as compared to control. The human milk zinc concentrations in the placebo group were 42.1 (31.1-51.7) and 16.8 (11.2-24.3) μmol/L at 1 month and 6 months postpartum respectively. The group supplemented with zinc alone had milk zinc concentrations of 49.3 (31.3-62.1) μmol/L at 1 month and 15.3 (11.8-27.2) μmol/L at 6 months while the group co-supplemented with beta-Carotene and zinc had breastmilk zinc concentrations of 46.7 (37.0-61.8) and 17.7 (12.9-27.4) μmol/L at 1 months and 6 months respectively. Thus, supplementation during pregnancy with zinc, either alone or combined with beta-carotene, had no effect on of zinc status 6 months postpartum (Dijkhuizen 2004).

While there have been reports of zinc supplementation slowing down the rate of decline in milk zinc concentration during lactation, thus far no correlation has been found between maternal zinc intake and milk zinc concentration. Similarly, no correlation has been found between maternal zinc intake and milk zinc concentration. (Dorea 2000, Domellof, Hemell et al. 2004, Hannan, Faraji et al. 2009, Donangelo and King 2012, 2014, Sabatier, Garcia-Rodenas et al. 2019).

Functions of zinc in general include its catalytic activity of approximately 100 enzymes; its role in immune function; its role in protein synthesis; its role in DNA synthesis; and its role in growth and development during pregnancy, childhood, and adolescence. Zn insufficiency during pregnancy may lead to growth restrictions in the infants. Zinc supplementation during pregnancy in women with relatively low plasma zinc concentrations and lower BMI was seen to be associated with infant birth weight and head circumference (Goldenberg, Tamura et al. 1995, Hess and King 2009).

Zinc deficiency in infants and children can lead to increased incidences of infectious diseases due to suppressed immune function. Persistent zinc deficiency can lead to growth stunting, poor appetite, irritability, susceptibility to infections. (Simmer and Thompson 1985, Ackland and Michalczyk 2016, Dror and Allen 2018).

Zn supplements (25 mg/day) during pregnancy until birth in women with BMI <26 kg/m2 had a significant effect associated with infant's birth weight and head circumferences ((Goldenberg, Tamura et al. 1995). Zn supplements (11 mg/L vs. 6.7 mg/L) for 6 months in Canadian very low birth weight infants (<1500 g) led to improved linear growth velocity (Friel 1993). Zn supplements (3 mg per day) for 6 months in Chile term infants born small for gestational age led to improved weight gain and length gain (Castillo-Durin 1995). Zn supplements (10 mg/day 6 days a week) for 6 months in Ethiopian stunted and non-stunted infants led to increased height (greater effect among stunted infants) and increased weight, lower incidence of morbidity from cough, diarrhoea, fever and vomiting among stunted infants (Umeta 2000). Zn supplements in f term infants led to marginal effects in growth (Radhakrishna 2013, Heinig 2006).

Zinc also relates to infant immunity. For example, meta-analysis concluded Zn supplements in children of developing world led to 18% reduced diarrhoea and lower rate of pneumonia infection (Black 2003). Zn supplements (10 mg/day) for 120 days in Indian infants aged 6-35 months led to decrease in percentage of infants with Zn insufficiency (<60 μg/dL), 45% reduction I incidence of acute lower respiratory infections (Sazawal 1998). Zn supplements (5 mg/day) from day 30-284 months of age in Indian infants small for gestational age led to decreased risk of death due to acute diarrhoea, 68% reduction in mortality (Sazawal 2001). Zn supplements in very low birth weight infants led to reduced risks of necrotising enterocolitis and mortality (Terrin 2013).

Zinc also relates to cognitive development of the infants. For example, meta-analysis showed that Zn supplements in infants led to some improvements in behavior and activity levels but inconclusive evidence (Bhatnagar 2001).

There was only one study that supplemented women during pregnancy and observed the effect on Vitamin D activity (VDA) during the first two months of lactation. The trial (in New Zealand) was a double-blinded placebo controlled trial where pregnant women were recruited at 27 weeks of gestation and assigned to either the placebo group, or a group who received daily oral vitamin D3 (1,000 IU), or a group who received two dosages of daily oral vitamin D3 (2000 IU) until 36 weeks of gestation (Wall 2015). The trial measured serum 25-hydroxyvitamin D [25(OH)D] at enrollment, at 36 weeks of gestation and cord blood and VDA at two weeks and two months post-partum. Seventy-five women provided human milk samples (44 women provided human milk samples at both 2 weeks and 2 months post-partum). The concentrations of vitamin D2, vitamin D3, 25(OH)D2, and 25(OH)D3 were measured in the human milk. Human milk VDA comprised of vitamin D3 and 25(OH) D3 since the concentrations of vitamin D2 and 25(OH)D2 were undetectable. At two weeks, the mean VDA was 52 IU/L in the placebo group, 51 IU/L (in the 1,000 IU group, and 74 IU/L in the 2,000 IU group; While at two months, the mean VDA was 45 IU/L, 43 IU/L and 58 IU/L, respectively. It was thus seen that maternal vitamin D supplementation during 27-36 weeks of pregnancy of 2,000 IU/d resulted in a higher VDA of breast milk ≥2 months postpartum (Wall 2015).

There is conflicting data to support the association of maternal status as well as maternal diet on the HM status of vitamin D. However, there is evidence to show that vitamin D at higher doses (1,000-6,400 IU/d) supplemented during lactation increases the concentration of vitamin D in human milk. Maternal obesity shows a negative association with human milk 25 (OH)D concentrations. (Dror 2018, Keikha 2021).

Functions of vitamin D in general include promoting calcium absorption in the gut and maintaining adequate serum calcium and phosphate concentrations to enable normal bone mineralization and to prevent hypocalcemic tetany. Vitamin D is also needed for bone growth and bone remodeling. Vitamin D sufficiency prevents rickets in children and osteomalacia in adults Together with calcium, vitamin D also helps protect older adults from osteoporosis. Other functions of vitamin D include reduction of inflammation and modulation of such processes as cell growth, neuromuscular and immune function, and glucose metabolism.

Severe vitamin D deficiency during gestation and early life is a primary cause of rickets in infants and children. In children, vitamin D deficiency is manifested as rickets, a disease characterized by a failure of bone tissue to become properly mineralized, resulting in soft bones and skeletal deformities (Elder 2014). In addition to bone deformities and pain, severe rickets can cause failure to thrive, developmental delay, hypocalcemic seizures, tetanic spasms, cardiomyopathy, and dental abnormalities (Munns 2016, Uday 2017)

Vitamin D is essential for the prevention of vitamin D deficiency rickets and its associated myopathy in children, however the evidence that either maternal or childhood vitamin D concentrations at higher levels prevent rickets and might influence bone or foetal growth, or bone mass in children, is limited and needs further research. (Pettifor 2011).

Vitamin D plays an important role in infant bone growth, immune system development, and brain development, but is present in low concentrations in breast milk. (Dror 2010). Vitamin D may result in a slight increase in length/height-for-age z-score (L/HAZ) (Huey 2021). Vitamin D may result in little to no difference in linear growth, stunting, hypercalciuria, or hypercalaemia, compared to placebo or no intervention (Huey 2021).

Meta analysis showed that maternal vitamin D deficiency during pregnancy is associated with small for gestational age (SGA) (Chen 2017). There is inconclusive evidence for links to infections, wheezing, eczema as well as neurocognitive outcomes (Dawodu 2013, Kovacs 2008). Observational studies have suggested possible links with respiratory infections, immunity, and autism; however, well-conducted, large interventional trials are lacking (Fiscaletti 2017).

The present disclosure provides prenatal supplement compositions comprising zinc and/or vitamin D for increasing breastmilk micronutrient levels.

### SUMMARY

The present disclosure includes the recognition that supplementing a subject before and during pregnancy with a nutritional composition/nutritional supplement comprising at least one compound selected from the group consisting of vitamin D, vitamin B₂, vitamin B₆, vitamin B₁₂, and zinc, for sustained increased levels of the at least one compound in the subject's breastmilk after birth, would represent a breakthrough for prenatal supplements. Indeed, the method of supplementing a subject before and during pregnancy with a nutrient mix containing vitamin D, vitamin B₂, vitamin B₆, vitamin B₁₂, and/or zinc, as disclosed herein, led to sustained increased levels of these compounds in breastmilk even up to 12 months after birth.

### BRIEF DESCRIPTION OF DRAWINGS

FIG. 1 is a CONSORT diagram for the collection of human milk (HM) samples in the NiPPeR study. Reasons for exclusion during the preconception phase have been published previously (Godfrey et al., Diabetes Care. 2021; 44(5):1091-9), while reasons for exclusion during pregnancy and birth in Singapore (SGP) and New Zealand (NZL) are provided in Supplementary Table 2. ^{‡} There were no HM samples collected in the United Kingdom (UK), so all participants from that site were excluded from this diagram. * Number of participants who provided at least one HM sample during the first 12 months of lactation. ^{†} Number of participants who provided at least one HM sample during the first 3 months of lactation.

FIG. 2 is a set of graphs showing Zinc concentrations in human milk of control (●) and intervention (▪) groups in the NiPPeR study during the first 3 months of lactation: (A) Overall, (B) Singapore, and (C) New Zealand. Data are the least-squares means (i.e. adjusted means) for each group, adjusted for visit, an interaction term (group*visit), study site, adherence, and maternal pre-pregnancy body mass index; error bars represent the respective 95% confidence intervals. **p<0.01 for the difference between intervention and control groups at a given time point.

FIG. 3 is a set of graphs showing mineral concentrations in human milk of control (●) and intervention (▪) groups in the NiPPeR study, during the first 3 months of lactation: (A) calcium, (B) copper, (C) iodine, (D) iron, (E) magnesium, (F) manganese, (G) phosphorus, (H) potassium, (I) selenium, and (J) sodium. Data are the least-squares means (i.e. adjusted means) for each group, adjusted for visit, an interaction term (group*visit), study site, adherence, and maternal pre-pregnancy body mass index; error bars represent the respective 95% confidence intervals. *p<0.05 for a difference between intervention and control groups at a given time point.

FIG. 4 is a set of graphs showing Zinc concentrations in human milk from control (●) and intervention (▪) groups in New Zealand in the NiPPeR study, during the first 12 months of lactation. Data are the least-squares means (i.e. adjusted means) for each group adjusted for visit, a group*visit interaction term, adherence, and maternal pre-pregnancy body mass index; error bars represent the respective 95% confidence intervals.

FIGS. 5A-5J is a set of graphs showing mineral concentrations in human milk from control (●) and intervention (▪) groups in New Zealand in the NiPPeR study, during the first 12 months of lactation: (A) calcium, (B) copper, (C) iodine, (D) iron, (E) magnesium, (F) manganese, (G) phosphorus, (H) potassium, (I) selenium, and (J) sodium. Data are the least-squares means (i.e., adjusted means) for each group adjusted for visit, a group*visit interaction term, adherence, and maternal pre-pregnancy body mass index; error bars represent the respective 95% confidence intervals. **p<0.01 for the difference between intervention and control at a given time point.

FIGS. 6A-6J is a set of graphs showing Mineral concentrations in human milk for the control (●) and intervention (▪) groups in Singapore in the NiPPeR study, during the first 3 months of lactation: (A) calcium, (B) copper, (C) iodine, (D) iron, (E) magnesium, (F) manganese, (G) phosphorus, (H) potassium, (I) selenium, and (J) sodium. Data are the least-squares means (i.e. adjusted means) for each group adjusted for visit, an interaction term (group*visit), adherence, and maternal pre-pregnancy body mass index; error bars represent the respective 95% confidence intervals. *p<0.05 for the difference between intervention and control at a given time point.

FIGS. 7A-7C show total vitamin D₃ concentrations in human milk of control and intervention groups in the NiPPeR study during the first 3 months of lactation: (A) Overall, (B) Singapore, and (C) New Zealand. Data are the least-squares means (i.e., adjusted means for each group, adjusted for visit, an interaction term (group*visit), study site, adherence, maternal pre-pregnancy body mass index, baseline serum vitamin D₃ concentrations and season; error bars represent the respective 95% confidence intervals. *p<0.05, **p<0.01 for the difference between intervention (closed square) and control groups (open circles) at a given time point.

FIGS. 8A-8C show vitamin D₃ concentrations in human milk of control and intervention groups in the NiPPeR study during the first 3 months of lactation: (A) Overall, (B) Singapore, and (C) New Zealand. Data are the least-squares means (i.e., adjusted means for each group, adjusted for visit, an interaction term (group*visit), study site, adherence, maternal pre-pregnancy body mass index, baseline serum vitamin D₃ concentrations and season; error bars represent the respective 95% confidence intervals. *p<0.05, **p<0.01 for the difference between intervention (closed square) and control (open circle) groups at a given time point.

FIGS. 9A-9C show vitamin 25(OH)D₃ concentrations in human milk of control and intervention groups in the NiPPeR study during the first 3 months of lactation: (A) Overall, (B) Singapore, and (C) New Zealand. Data are the least-squares means (i.e., adjusted means for each group, adjusted for visit, an interaction term (group*visit), study site, adherence, maternal pre-pregnancy body mass index, baseline serum vitamin D₃ concentrations and season; error bars represent the respective 95% confidence intervals. *p<0.05, **p<0.01 for the difference between intervention (closed square) and control (open circle) groups at a given time point.

FIGS. 10A-10E show vitamin B concentrations in human milk of control and intervention groups in the NiPPeR study, during the first 3 months of lactation: (A) vitamin B₁, (B) vitamin B₂, (C) vitamin B₃, (D) vitamin B₆, (E) vitamin B₉. Data are the lest-squares means (i.e. adjusted means) for each group, adjusted for visit, an interaction term (group*visit), study site, adherence, infant gestational age, and maternal pre-pregnancy body mass index; error bars represent the respective 95% confidence intervals. *p<0.05 for a difference between intervention (closed square) and control (open circle) groups at a given time point.

FIGS. 11A-11F show vitamin concentrations in human milk from control and intervention groups in New Zealand in the NiPPeR Study, during the first 12 months of lactation: (A) total vitamin D₃, (B) vitamin B₁, (C) vitamin B₂, (D) vitamin B₃, (E) vitamin B₆, (F) vitamin B₉. Data are the least-squares means (i.e. adjusted means) for each group adjusted for visit, a group*visit interaction term, adherence, maternal pre-pregnancy body mass index, baseline serum vitamin D₃ concentrations and season for (A), and adjusted for visit, a group*visit interaction term, adherence, infant gestational age and maternal pre-pregnancy body mass index for (B)˜(F); error bars represent the respective 95% confidence intervals. *p<0.05, **p<0.01 for the difference between intervention (closed square) and control (open circle) groups at a given time point.

FIGS. 12A-12E show the average contribution (%) of human milk B-vitamers in New Zealand in the NiPPeR study, during the first 12 months of lactation for (A) vitamin B₁, (B) vitamin B₂, (C) vitamin B₃ (D) vitamin B₆ and (E) vitamin B₉. Data represents the mean contribution of each vitamer at a given visit; error bars represent the respective 95% confidence intervals. Abbreviations: 5MeTHF, 5-methyl tetrahydrofolic acid; FAD, flavin adenine dinucleotide; FMN, flavin mononucleotide; NMN, nicotinamide mononucleotide; NRT, nicotinamide riboside; PLP, pyridoxal 5′-phosphate; PMP, pyridoxamine-5′-phosphate; TMP, thiamine monophosphate; TPP, thiamine pyrophosphate.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one dithionite or a functionally similar reducing agent” should be interpreted as “dithionite,” or “a functionally similar reducing agent,” or “both dithionite and a functionally similar reducing agent.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The term “subject,” as used herein, refers to a mammal. Mammal includes, but is not limited to, rodents, aquatic mammals, domestic animals such as dogs and cats, farm animals such as sheep, pigs, cows and horses, and humans. In one embodiment, the mammal may be a cat, a dog or a human. The human may be a woman, for example, a woman who is trying to get pregnant, or who is pregnant. In one embodiment of the invention, the subject is a mammal selected from the group consisting of a cat, a dog and, a human. For example, the subject may a woman who is trying to get pregnant, or who is pregnant.

The term “probiotic,” as used herein, refers to live probiotic bacteria, non-replicating probiotic bacteria, dead probiotic bacteria, non-viable probiotic bacteria, fragments of probiotic bacteria such as DNA, metabolites of probiotic bacteria, cytoplasmic compounds of probiotic bacteria, cell wall materials of probiotic bacteria, culture supernatants of probiotic bacteria, and/or combinations of any of the foregoing. The probiotic may for example be live probiotic bacteria, non-replicating probiotic bacteria, dead probiotic bacteria, non-viable probiotic bacteria, or any combination thereof. In an embodiment of the invention the probiotic is live probiotic bacteria.

In one embodiment, the probiotics in the compositions disclosed herein may comprise at least one of or a combination of Lactobacillus and Bifidobacterium. The most preferred Lactobacillus strain is the Lactobacillus rhamnosus GG strain available under the deposit number CGMCC 1.3724. The most preferred Bifidobacterium strain is the Bifidobacterium lactis BB12 strain deposited as CNCM 1-3446. Preferably the probiotics may comprise at least one of or a mixture of the Lactobacillus rhamnosus GG strain available under the deposit number CGMCC 1.3724 and of the Bifidobacterium lactis BB12 strain deposited as CNCM 1-3446. Most preferably the probiotics may consist of a mixture of the Lactobacillus rhamnosus GG strain available under the deposit number CGMCC 1.3724 and of the Bifidobacterium lactis BB12 strain deposited as CNCM 1-3446.

The term “myo-inositol” or “cis-1,2,3,5-trans-4,6-cyclohexanehexol,” as used herein, refers to a predominant isomeric form of inositol. Myo-inositol is a compound present in animal and plant cells and plays an important role in various cellular processes, as the structural basis for secondary messengers in eukaryotic cells, in particular as inositol triphosphates (IP3), phosphatidylinositol phosphate lipids (PIP2/PIP3) and inositol glycans. Myo-inositol has been shown to participate in a variety of biological process such as cell growth and survival, development and function of peripheral nerves, osteogenesis, energy metabolism and reproduction (Craze et al., (2013) Biochimie 95:1811-1827). Myo-inositol is found as free form, phosphoinositides and phytic acid, in fresh fruits and vegetables, and in all foods containing seeds (beans, grains and nuts) (Clements R S and Darnell B., Am J Clin Nutr (1980) 33:1954-1967). Myo-inositol from phytic acid can be released in the gut by phytases found in plants, microorganisms and in animal tissues (Schlemmer U et al., Mol Nutr Food Res (2009) 53:S330-S375). These enzymes are capable of releasing free inositol, orthophosphate, and intermediary products including the mono-, di-, tri-, tetra- and penta-phosphate forms of inositol. Much of the ingested inositol hexaphosphate is hydrolysed to inositol. Myo-inositol is also commercially available from several suppliers.

Myo-inositol may be administered in accordance with the present disclosure in any effective amount. Typically, an effective amount will depend on the type, age, size, health status, lifestyle and/or genetic heritage of the subject. The effective amount may be split into several smaller amounts and administered throughout the day so as the total daily intake is the effective amount. A person skilled in the art will be able to propose appropriate amounts of myo-inositol to be consumed per day. For example, the composition for use in accordance with the present disclosure may be administered in a daily dose comprising myo-inositol in an amount of 0.2 to 11 g, 0.2 to 5 g, 0.4 to 10 g, 0.6 to 9 g, 0.8 to 8 g, 1.0 to 7 g, 1.2 to 6.5 g, preferably 1.5 to 6 g, more preferably 2 to 5.5 g, more preferably 3 to 5 g, even more preferably 4 g.

The term “vitamin B,” as used herein, refers to one or more of thiamin (vitamin B1), riboflavin (vitamin B2), niacin (vitamin B3), pantothenic acid (vitamin B5), pyridoxine (vitamin B6), biotin (vitamin B7), myo-inositol (vitamin B8), folic acid (vitamin B9), cobalamin (vitamin B12), including salts, esters or derivatives thereof. In one embodiment, vitamin B of the present disclosure comprises one or more of riboflavin (vitamin B2), pyridoxine (vitamin B6) and cobalamin (vitamin B12), including salts, esters or derivatives thereof.

For example, the nutritional composition of the present disclosure may further comprise a combination of vitamins. In one embodiment, the nutritional composition may comprise at least one vitamin B selected from the group consisting of vitamin B2, vitamin B6, and vitamin B12 and mixtures thereof. For example, the composition comprises vitamin B2, vitamin B6 and vitamin B12.

Pregnant women may also be more often deficient in vitamins B2, B6, B12 compared to other nutrients. Also, vitamin B2, B6, B12 might not be consumed in sufficient amounts by a significant proportion of the pregnant woman population. It is therefore of particular interest to supplement the diet of pregnant women with these vitamins in order to compensate these particularly often-occurring deficiencies.

The term “vitamin D,” as used herein, refers to a group of fat-soluble secosteroids responsible for increasing intestinal absorption of calcium, magnesium, and phosphate, and many other biological effects. Vitamin D comprises vitamin D₁ (Mixture of molecular compounds of ergocalciferol with lumisterol, 1:1), vitamin D₂ (ergocalciferol; made from ergosterol), vitamin D₃ (cholecalciferol; made from 7-dehydrocholesterol in the skin), vitamin D₄ (22-dihydroergocalciferol), and vitamin D₅ (sitocalciferol; made from 7-dehydrositosterol). In humans, the most important compounds in this group are vitamin D₃ (also known as cholecalciferol) and vitamin D₂ (ergocalciferol).

The term “zinc,” as used herein, refers to the chemical element with the symbol Zn and atomic number 30. In one embodiment, zinc may be included in the present disclosure with forms as zinc oxide, zinc acetate, zinc gluconate, zinc sulfate, zinc citrate, or picolinate.

The term “nutritional composition,” or “nutritional supplement,” as used herein, refers to a nutritional product that provides nutrients to an individual that may otherwise not be consumed in sufficient quantities by the individual. For instance, a nutritional composition or nutritional supplement of the present disclosure may include vitamins, minerals, fiber, fatty acids, or amino acids. Nutritional compositions or nutritional supplements of the present disclosure may for example be provided in the form of a pill, a tablet, a lozenge, a chewy capsule or tablet, a tablet or capsule, or a powder supplement that can for example be dissolved in water or sprinkled on food.

In one embodiment, nutritional compositions or nutritional supplements of the present disclosure may provide selected nutrients while not representing a significant portion of the overall nutritional needs of a subject. Typically, they do not represent more than 0.1%, 1%, 5%, 10% or 20% of the daily energy need of a subject. A nutritional composition or nutritional supplement of the present disclosure may be used during pregnancy, e.g., as a maternal supplement.

As used herein, an “effective amount” is an amount that prevents a deficiency, treats a disease or medical condition in an individual or, more generally, reduces symptoms, manages progression of the diseases or provides a nutritional, physiological, or medical benefit to the individual. The relative terms “promote,” “improve,” “increase,” “enhance” and the like refer to the effects of a nutritional product comprising vitamin D and/or zinc disclosed herein relative to a nutritional product lacking the vitamin D and/or zinc, but otherwise identical.

The term “unit dosage form,” as used herein, refers to physically discrete units suitable as unitary dosages for human and animal subjects, each unit containing a predetermined quantity of the nutritional composition disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of powder in a sachet.

The term “nutritional product,” as used herein, refers to any product that can be used to provide nutrition to a subject. Typically, a nutritional product contains a protein source, a carbohydrate source and a lipid source.

The term “food product,” as used herein, refers to any kind of product that may be safely consumed by a human or an animal. A food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The food product may also contain anti-oxidants, stabilizers (when provided in solid form) or emulsifiers (when provided in liquid form).

The term “functional food product,” as used herein, refers to a food product providing an additional health-promoting or disease-preventing function to the individual.

The term “healthy ageing product,” as used herein, refers to a product providing an additional health-promoting or disease-preventing function related to healthy ageing to the individual.

The term “dairy products,” as used herein, refers to food products produced from milk or fractions of milk from animals such as cows, goats, sheep, yaks, horses, camels, and other mammals. Examples of dairy products are low fat milk (e.g., 0.1%, 0.5% or 1.5% fat), fat-free milk, milk powder, whole milk, whole milk products, butter, buttermilk, buttermilk products, skim milk, skim milk products, high milk-fat products, condensed milk, creme fraiche, cheese, ice cream and confectionery products, probiotic drinks or probiotic yoghurt type drinks.

The term “dairy alternative product,” as used herein, refers to products similar to dairy products but produced without milk.

The term “milk,” as used herein, is defined by Codex Alimentarius as the normal mammary secretion of milking animals obtained from one or more milkings without either addition to it or extraction from it, intended for consumption as liquid milk or for further processing.

The term “beverage product,” as used herein, refers to a nutritional product in liquid or semi-liquid form that may be safely consumed by an individual.

The term “diet product,” as used herein, refers to a food product with a restricted and/or reduced caloric content.

The term “pet food product,” as used herein, refers to a nutritional product that is intended for consumption by pets. A pet, or companion animal, as referenced herein, is to be understood as an animal selected from dogs, cats, birds, fish, rodents such as mice, rats.

All ingredients of the composition can be admixed together or alternatively the composition can be provided in the form of a kit of parts wherein ingredients or groups of ingredients are provided separately. These separate compositions may be intended to be consumed separately or together.

### EMBODIMENTS

An aspect of the present disclosure is a method of supplementing a subject before and during pregnancy with a nutrient mix containing Vitamin D and/or zinc to sustain increased levels of vitamin D and/or zinc in breastmilk up to 12 months after birth, once the supplementing stops after the subject gives birth.

Applicant surprisingly found that supplementing a subject before and during pregnancy with a nutritional composition/nutritional supplement comprising vitamin D and/or zinc can lead to sustained increased levels of vitamin D and/or zinc in the subject's breastmilk up to 12 months after birth when the supplementing stops after the subject gives birth. For example, Applicant surprisingly found that supplementing a subject before and during pregnancy with a nutritional composition/nutritional supplement comprising vitamin D can lead to about 44% to about 102% overall increase of the vitamin D level in the subject's breastmilk up to 12 months after birth when the supplementing stops after the subject gives birth. Applicant further surprisingly found that supplementing a subject before and during pregnancy with a nutritional composition/nutritional supplement comprising zinc can lead to about 10% overall increase of the zinc level in the subject's breastmilk up to 12 months after birth when the supplementing stops after the subject gives birth.

In an aspect, the present disclosure relates to a method for increasing at least one micronutrient level in a subject's breastmilk. In one embodiment, the at least one micronutrient comprises at least one of vitamin D and zinc. In one embodiment, the at least one micronutrient is vitamin D. In another embodiment, the at least one micronutrient is zinc. In another embodiment, the at least one micronutrient comprises both vitamin D and zinc.

The subject is a mammal such as a cat, a dog or a human. In one embodiment, the subject is a female mammal who is trying to get pregnant, or who is pregnant. In one preferred embodiment, the subject is a female woman who is trying to get pregnant, or who is pregnant.

In one embodiment, the method for increasing at least one micronutrient level (e.g., vitamin D and/or zinc level) in a subject's breastmilk comprises administering to the subject a nutritional composition or nutritional supplement comprising at least one of vitamin D and zinc.

In one embodiment, the nutritional composition or nutritional supplement comprises either vitamin D or zinc. In another embodiment, the nutritional composition or nutritional supplement comprises both vitamin D and zinc.

The nutritional composition or nutritional supplement may be administered to provide an effective amount of vitamin D and/or zinc. Typically, an effective amount may depend on the type, age, size, health status, lifestyle and/or genetic heritage of the subject. The effective amount may be split into several smaller amounts and administered throughout the day so as the total daily intake is the effective amount.

In one embodiment, the nutritional composition or nutritional supplement may be administered in a daily dose comprising vitamin D in an amount of between about 1.5 μg and about 100 μg, between about 1.8 μg and about 96.5 μg, between about 2.1 μg and about 93 μg, between about 2.4 μg and about 89.5 μg, between about 2.7 μg and about 86 μg, between about 3 μg and about 82.5 μg, between about 3.3 μg and about 79 μg, between about 3.6 μg and about 75.5 μg, between about 3.9 μg and about 72 μg, between about 4.2 μg and about 68.5 μg, between about 4.5 μg and about 65 μg, between about 4.8 μg and about 61.5 μg, between about 5.1 μg and about 58 μg, between about 5.4 μg and about 54.5 μg, between about 5.7 μg and about 51 μg, between about 6 μg and about 47.5 μg, between about 6.3 μg and about 44 μg, between about 6.6 μg and about 40.5 μg, between about 6.9 μg and about 37 μg, between about 7.2 μg and about 33.5 μg, between about 7.5 μg and about 30 μg, between about 7.8 μg and about 26.5 μg, between about 8.1 μg and about 23 μg, between about 8.4 μg and about 19.5 μg, between about 8.7 μg and about 16 μg, between about 9.0 μg and about 12.5 μg, between about 9.3 μg and about 11.5 μg, preferably about 10 μg.

In one embodiment, the nutritional composition or nutritional supplement may be administered in a daily dose comprising zinc in an amount of between about 1.1 mg and about 40 mg, between about 1.4 mg and about 38.8 mg, between about 1.7 mg and about 37.6 mg, between about 2.0 mg and about 36.4 mg, between about 2.3 mg and about 35.2 mg, between about 2.6 mg and about 34 mg, between about 2.9 mg and about 32.8 mg, between about 3.2 mg and about 31.6 mg, between about 3.5 mg and about 30.4 mg, between about 3.8 mg and about 29.2 mg, between about 4.1 mg and about 28 mg, between about 4.4 mg and about 26.8 mg, between about 4.7 mg and about 25.6 mg, between about 5.0 mg and about 24.4 mg, between about 5.3 mg and about 23.2 mg, between about 5.8 mg and about 22 mg, between about 6.1 mg and about 20.8 mg, between about 6.4 mg and about 19.6 mg, between about 6.7 mg and about 18.4 mg, between about 7.0 mg and about 17.2 mg, between about 7.3 mg and about 16 mg, between about 7.6 mg and about 14.8 mg, between about 7.9 mg and about 13.6 mg, between about 8.2 mg and about 12.4 mg, between about 8.5 mg and about 11.2 mg, between about 8.8 mg and about 6 mg, between about 9.1 mg and about 11.0 mg, between about 9.4 mg and about 10.8 mg, between about 9.7 mg and about 10.4 mg, preferably about 10 mg per daily dose.

In one embodiment, the nutritional composition or nutritional supplement may be administered in a daily dose comprising vitamin D in an amount of between about 1.5 μg vitamin D and about 100 μg and zinc in an amount of between about 1.1 mg and about 40 mg. In one preferred embodiment, may be administered in a daily dose comprising about 10 μg vitamin D and about 10 mg zinc.

In one embodiment, the nutritional composition or nutritional supplement comprising vitamin D and/or zinc may be used for maternal administration. For example, the nutritional composition or nutritional supplement comprising vitamin D and/or zinc may be administered to a female mammal who is desiring to get pregnant, or to a pregnant female mammal. Preferably, the nutritional composition or nutritional supplement comprising vitamin D and/or zinc may be administered to a woman who is desiring to get pregnant, or to a pregnant woman, preferably, to a pregnant woman.

The nutritional composition or nutritional supplement comprising vitamin D and/or zinc of the present disclosure may be administered to a woman desiring to get pregnant, for example during at least 1, 2, 3 or 4 months preceding the pregnancy or desired pregnancy. When the nutritional composition or nutritional supplement comprising vitamin D and/or zinc may be administered to a pregnant woman, the nutritional composition or nutritional supplement comprising vitamin D and/or zinc may be preferably administered for at least 4, preferably at least 8, more preferably at least 12, more preferably at least 16, more preferably at least 20, more preferably at least 24, more preferably at least 28, even more preferably at least 36 weeks during pregnancy. As the nutritional requirements increase in the second and third trimester of pregnancy, it is further preferred to administer the nutritional composition or nutritional supplement comprising vitamin D and/or zinc of the present disclosure throughout the third trimester of pregnancy and most preferably throughout the second and third trimesters of pregnancy.

In one embodiment, the nutritional composition or nutritional supplement comprising vitamin D and/or zinc is administered to the subject until the subject gives birth to a baby (or babies). Specifically, the subject stops taking the nutritional composition or nutritional supplement comprising vitamin D and/or zinc once the subject gives birth to a baby (or babies).

As shown in Example 1, the subjects were administered with the nutritional composition or nutritional supplement comprising both vitamin D in an amount of about 10 μg per daily dose and zinc in an amount of about 10 mg per daily dose. The administration of the nutritional composition or nutritional supplement stopped when the subjects gave birth. Example 1 surprisingly showed that there was an about 44% overall increase of the vitamin D level in the subject's breastmilk up to 12 months after birth as compared with the control subjects who were not administered with the nutritional composition or nutritional supplement comprising vitamin D of the present disclosure.

Further, Example 1 surprisingly showed that there was an about 10% overall increase of the zinc level in the subject's breastmilk up to 12 months after birth as compared with the control subjects who were not administered with the nutritional composition or nutritional supplement comprising zinc of the present disclosure.

In one embodiment, the nutritional composition or nutritional supplement of the present disclosure can lead to at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149% or 150% increase of the vitamin D or zinc level as compared to the control subjects who were not administered with the nutritional composition or nutritional supplement comprising vitamin D or zinc of the present disclosure.

In one embodiment, the increase of the vitamin D or zinc level can be sustained up to 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 36 months, or 48 months.

Thus, the experimental data of Example 1 showed that administering the nutritional composition or nutritional supplement comprising vitamin D and/or zinc starting from before pregnancy but stopped at birth can have an impact on the vitamin D or zinc level of the breastmilk of the subject. For example, the experimental data of Example 1 showed that increase of vitamin D and zinc levels in breastmilk can be sustained for 12 months even after stopping the intervention. As both vitamin D and zinc are important for the infant development, e.g., for its immune system, increased levels of in vitamin D and zinc breastmilk can be beneficial for breastfed infants for which current recommendation is to supplement with vitamin D and zinc after birth.

In one embodiment, the nutritional composition or nutritional supplement further comprises myo-inositol. For example, the nutritional composition or nutritional supplement may be administered in a daily dose comprising myo-inositol in an amount of between about 0.2 g and about 5 g, preferably between about 1.5 g and about 5 g, more preferably between about 2 g and about 5 g, most preferably between about 2 g and about 4 g. In one preferred embodiment, the nutritional composition or nutritional supplement may be administered in a daily dose comprising about 4 g myo-inositol.

In one embodiment, the nutritional composition or nutritional supplement further comprises probiotics.

For example, the probiotics may comprise a combination of Lactobacillus and Bifidobacterium. In one embodiment, the Lactobacillus strain may be the Lactobacillus rhamnosus GG strain available under the deposit number CGMCC 1.3724. The Bifidobacterium strain may be the Bifidobacterium lactis BB12 strain deposited as CNCM 1-3446. In one preferred embodiment, the probiotics comprises a mixture of the Lactobacillus rhamnosus GG strain available under the deposit number CGMCC 1.3724 and the Bifidobacterium lactis BB12 strain deposited as CNCM 1-3446. In one even more preferred embodiment, the probiotics consists of a mixture of the Lactobacillus rhamnosus GG strain available under the deposit number CGMCC 1.3724 and the Bifidobacterium lactis BB12 strain deposited as CNCM 1-3446.

In one embodiment, the probiotic is provided in an amount of between about 10⁵ and about 10¹² cfu per daily dose, between about 10⁶ and about 10^{11.5} cfu per daily dose, more preferably between about 10⁷ and about 10¹¹ cfu per daily dose, most preferably about 10⁹ cfu per daily dose.

The nutritional composition or nutritional supplement may further comprise a combination of vitamin Bs. For example, the nutritional composition or nutritional supplement may comprise at least one vitamin B selected from the group consisting of vitamin B2, vitamin B6, vitamin B12, and mixtures thereof. Preferably, the nutritional composition or nutritional supplement comprises vitamin B2, vitamin B6 and vitamin B12.

In one embodiment, pregnant women may be more often deficient in vitamins B6 and B12 compared to other nutrients. Also, vitamin B2 may not be consumed in sufficient amounts by a significant proportion of the pregnant woman population. It is therefore of particular interest to supplement the diet of pregnant women with these vitamin Bs in order to compensate these particularly often-occurring deficiencies.

In one embodiment, the nutritional composition or nutritional supplement may be administered in a daily dose comprising vitamin B2 in an amount of between about 0.14 mg and about 14 mg, between about 0.18 mg and about 13 mg, between about 0.22 mg and about 12 mg, between about 0.26 mg and about 11 mg, between about 0.3 mg and about 10 mg, between about 0.34 mg and about 9 mg, between about 0.38 mg and about 8 mg, between about 0.42 mg and about 7 mg, between about 0.46 mg and about 6 mg, between about 0.5 mg and about 5 mg, between about 0.54 mg and about 4 mg, between about 0.58 mg and about 3.6 mg, between about 0.62 mg and about 3.2 mg, between about 0.66 mg and about 2.8 mg, between about 0.7 mg and about 2.4 mg, between about 0.8 mg and about 2.3 mg, between about 0.9 mg and about 2.2 mg, between about 1.0 mg and about 2.1 mg, between about 1.1 mg and about 2.0 mg, between about 1.2 mg and about 1.95 mg, between about 1.3 mg and about 1.92 mg, between about 1.4 mg and about 1.90 mg, between about 1.5 mg and about 1.89 mg, between about 1.6 mg and about 1.88 mg, between about 1.7 mg and about 1.86 mg, between about 1.75 mg and about 1.82 mg, between about 1.78 mg and about 1.81 mg, preferably about 1.8 mg.

In one embodiment, the nutritional composition or nutritional supplement may be administered in a daily dose comprising vitamin B6 in an amount of between about 0.19 mg and about 19 mg, between about 0.28 mg and about 18.4 mg, between about 0.37 mg and about 17.8 mg, between about 0.46 mg and about 17.2 mg, between about 0.55 mg and about 16.6 mg, between about 0.64 mg and about 16 mg, between about 0.73 mg and about 15.4 mg, between about 0.82 mg and about 16.8 mg, between about 0.91 mg and about 16.2 mg, between about 1 mg and about 15.6 mg, between about 1.09 mg and about 15 mg, between about 1.18 mg and about 14.4 mg, between about 1.27 mg and about 13.8 mg, between about 1.36 mg and about 13.2 mg, between about 1.45 mg and about 12.6 mg, between about 1.54 mg and about 12 mg, between about 1.63 mg and about 11.4 mg, between about 1.72 mg and about 10.8 mg, between about 1.81 mg and about 10.2 mg, between about 1.9 mg and about 9.6 mg, between about 1.99 mg and about 9 mg, between about 2.08 mg and about 8.4 mg, between about 2.17 mg and about 7.8 mg, between about 2.26 mg and about 7.2 mg, between about 2.35 mg and about 6.6 mg, between about 2.44 mg and about 6 mg, between about 2.53 mg and about 4.4 mg, between about 2.54 mg and about 3.8 mg, between about 2.55 mg and about 3.2 mg, preferably about 2.6 mg.

In one embodiment, the nutritional composition or nutritional supplement may be administered in a daily dose comprising vitamin B12 in an amount of between about 0.26 μg and about 26 μg, between about 0.45 μg and about 25.2 μg, between about 0.64 μg and about 24.4 μg, between about 0.83 μg and about 23.6 μg, between about 1.02 μg and about 22.8 μg, between about 1.21 μg and about 22 μg, between about 1.4 μg and about 21.2 μg, between about 1.59 μg and about 20.4 μg, between about 1.78 μg and about 19.6 μg, between about 1.97 μg and about 18.8 μg, between about 2.16 μg and about 18 μg, between about 2.35 μg and about 17.2 μg, between about 2.54 μg and about 16.4 μg, between about 2.73 μg and about 15.6 μg, between about 2.92 μg and about 14.8 μg, between about 3.11 μg and about 14 μg, between about 3.3 μg and about 13.2 μg, between about 3.49 μg and about 12.4 μg, between about 3.68 μg and about 11.6 μg, between about 3.87 μg and about 10.8 μg, between about 4.06 μg and about 10 μg, between about 4.25 μg and about 9.2 μg, between about 4.44 μg and about 8.4 μg, between about 4.63 μg and about 7.6 μg, between about 4.82 μg and about 6.8 μg, between about 5.01 μg and about 6 μg, between about 5.1 μg and about 5.4 μg, preferably about 5.2 μg.

In one preferred embodiment, the nutritional composition or nutritional supplement may be administered in a daily dose comprising vitamin B2 in an amount of between about 0.14 mg and about 14 mg, preferably about 1.8 mg; vitamin B6 in an amount of between about 0.19 mg and about 19 mg, preferably about 2.6 mg; and vitamin B12 in an amount of between about 0.26 μg and about 26 μg, preferably about 5.2 μg. More preferably, the nutritional composition or nutritional supplement may be administered in a daily dose comprising about 1.8 mg vitamin B2, about 2.6 mg vitamin B6, and about 5.2 μg vitamin B12.

In some embodiments, the nutritional composition comprises one or more of Vitamin B₁, B₂, B₃ and is administered to the subject such that breastmilk levels increase during at least the time period from the first week of lactation through the sixth week of lactation. In some embodiments, the nutritional composition comprises Vitamin B₆ and is administered to the subject such that breastmilk levels increase during at least the time period from the first week of lactation through the third month of lactation.

In one embodiment, the nutritional composition or nutritional supplement comprises vitamin D, zinc, myo-inositol, vitamin B2, vitamin B6, vitamin B12, Bifidobacterium lactis BB12 CNCM1-3446 and Lactobacillus rhamnosus GG CGMCC 1.3724. For example, the nutritional composition or nutritional supplement may be administered in a daily dose comprising between about 1.5 μg and about 100 μg of vitamin D, between about 1.1 mg and about 40 mg of zinc, between about 0.2 g and about 11 g (preferably between about 0.2 g and about 5 g) of myo-inositol, between about 0.14 mg and about 14 mg of vitamin B2, between about 0.19 mg and about 19 mg of vitamin B6, between about 0.26 μg and about 26 μg of vitamin B12, between about 10⁶ and about 10^{11.5} cfu of a mixture of Bifidobacterium lactis BB12 CNCM1-3446 and Lactobacillus rhamnosus GG CGMCC 1.3724.

In another embodiment, the nutritional composition or nutritional supplement may be administered in a daily dose comprising about 10 μg vitamin D, about 10 mg zinc, about 4 g myo-inositol, about 1.8 mg vitamin B2, about 2.6 mg B6, about 5.2 μg B12 and about 10⁹ cfu of a mixture of Bifidobacterium lactis BB12 CNCM1-3446 and Lactobacillus rhamnosus GG CGMCC 1.3724.

The nutritional composition or nutritional supplement may further comprise other vitamins, minerals and nutrients. For example, additional vitamins, minerals and nutrients may be included according to the recommended standards such as “U.S. Recommended Daily Allowances” (USRDA) and those of other government bodies.

In one embodiment, the nutritional composition or nutritional supplement may further comprise one or more of calcium, magnesium, phosphorus, iron, copper, iodine, selenium, vitamin A or retinal activity equivalent (RAE) for example in the form of beta carotene or a mix of carotenoids, Vitamin C, Vitamin B1, niacin, folic acid, biotin, Vitamin E.

In one embodiment, the nutritional composition or nutritional supplement may further comprise calcium, iron, iodine, folic acid, and vitamin A or retinal activity equivalent (RAE) for example in the form of beta carotene or a mix of carotenoids. In one embodiment, the nutritional composition or nutritional supplement may further comprise calcium, iron, iodine, folic acid, and beta carotene.

For example, the nutritional composition or nutritional supplement may be administered in a daily dose comprising at least one of the following: between about 10 mg and about 2500 mg, about 100 mg and about 2500 mg, preferably between about 50 mg and about 500 mg, more preferably between about 100 mg and about 200 mg, more preferably about 150 mg of calcium; between about 35 mg and about 350 mg, preferably between about 50 mg and about 250 mg, more preferably between about 75 mg and about 150 mg, more preferably about 120 mg of magnesium; between about 70 mg and about 3500 mg, between about 150 mg and about 700 mg, between about 220 mg and about 500 mg, between about 300 mg and about 400 mg, more preferably about 350 mg of phosphorus; between about 2.7 mg and about 45 mg, preferably between about 5.0 mg and about 30 mg, between about 7.5 mg and about 15 mg, between about 10.5 mg and about 13.5 mg, more preferably about 12 mg of iron; between about 0.1 mg and 10 mg, preferably between about 0.5 mg and 5 mg, between about 1.0 mg and 3 mg, between about 1.5 mg and 2 mg of copper; between about 22 μg and about 1,100 μg, preferably between about 50 μg and about 550 μg, between about 100 μg and about 300 μg, between about 120 μg and about 180 μg, more preferably about 150 μg of iodine; between about 6 μg and about 400 μg, preferably between about 12 μg and about 200 μg, between about 25 μg and about 150 μg, between about 50 μg and about 100 μg of selenium; between about 77 μg and about 3000 μg, preferably between about 80 μg and about 1500 μg, between about 90 μg and about 500 μg, between about 100 μg and about 200 μg, more preferably about 120 μg of vitamin A or retinal activity equivalent (RAE) for example in the form of beta carotene or a mix of carotenoids; between about 8.5 mg and about 850 mg, preferably between about 17 mg and about 500 mg, between about 35 mg and about 250 mg, between about 50 mg and about 150 mg of Vitamin C; between about 0.14 mg and about 14 mg, preferably between about 0.28 mg and about 7 mg, between about 0.45 mg and about 5 mg, 0.9 mg and about 3 mg of Vitamin B1; between about 1.8 mg and about 35 mg, preferably between about 3.5 mg and about 20 mg, between about 5 mg and about 10 mg of niacin; between about 60 μg and about 1000 μg, preferably between about 120 μg and about 800 μg, between about 180 μg and about 600 μg, between about 240 μg and about 500 μg, between about 300 μg and about 450 μg, more preferably about 400 μg of folic acid; between about 3 μg and about 300 μg, preferably between about 10 μg and about 150 μg, between 20 μg and about 100 μg, between 40 μg and about 50 μg of biotin; or between about 1.9 μg and about 109 μg, preferably between about 3.8 μg and about 59 μg, between about 5.5 μg and about 39 μg, between about 10 μg and about 19 μg of Vitamin E.

In one embodiment, the nutritional composition or nutritional supplement may be administered in a daily dose comprising at least one of the following: between about 10 mg and about 2500 mg (preferably between about 50 mg and about 500 mg, more preferably between about 100 mg and about 200 mg, most preferably about 150 mg) of calcium; between about 2.7 mg and about 45 mg (preferably between about 5.0 mg and about 30 mg, between about 7.5 mg and about 15 mg, between about 10.5 mg and about 13.5 mg, more preferably about 12 mg) of iron, between about 22 μg and about 1,100 μg (preferably between about 50 μg and about 550 μg iodine, between about 100 μg and about 300 μg, between about 120 μg and about 180 μg, more preferably about 150 μg) of iodine; between about 60 μg and about 1000 μg (preferably between about 120 μg and about 800 μg, between about 180 μg and about 600 μg, between about 240 μg and about 500 μg, between about 300 μg and about 450 μg, more preferably about 400 μg) of folic acid; or between about 77 μg and about 3000 μg (preferably between about 80 μg and about 1500 μg, between about 90 μg and about 500 μg, between about 100 μg and about 200 μg, more preferably about 120 μg) of vitamin A or retinal activity equivalent (RAE) for example in the form of beta carotene or a mix of carotenoids.

In one embodiment, the nutritional composition or nutritional supplement may be administered in a daily dose comprising at least one of about 150 mg calcium, about 12 mg iron, about 150 μg iodine, about 400 μg folic acid, or about 120 μg vitamin A or retinal activity equivalent (RAE) for example in the form of beta carotene or a mix of carotenoids.

Long chain polyunsaturated fatty acids are recommended at an early stage of life of a baby and usually provided as an accompanying product or embedded in a product. In one embodiment, the nutritional composition or nutritional supplement of the present disclosure may advantageously further comprise a long chain polyunsaturated fatty acid, such as arachidonic acid (ARA), eicosapentaenoic acid (EPA) and/or docosahexaenoic acid (DHA), in a suitable amount as known by the person skilled in the art, for example in an amount of between about 100 mg and about 500 mg per daily dose, more preferably between about 200 and about 400 mg per daily dose. The fatty acid can be derived from fish oil, krill, poultry, eggs, a plant source, algae and/or a nut source, e.g., flax seed, walnuts, almonds.

Each of the components in the nutritional composition or nutritional supplement of the present disclosure may be used in any amount that is effective in achieving the objective of the present disclosure (i.e., increasing breastmilk micronutrient levels of a subject after the subject gives birth). For example, the skilled artisan would be able to determine appropriate dosages depending on age, size and health status of each specific subject, on her lifestyle, as well as on her genetic heritage.

In one embodiment, the amounts used in the present application are the daily dose. The amount of each component may be used as disclosed or changed (e.g., increased or decreased) depending on age, size and health status of each specific subject, on her lifestyle, as well as on her genetic heritage. In one embodiment, the nutritional composition or nutritional supplement of the present disclosure may be administered regularly, for example two times a day, daily, every two days or weekly.

In an aspect, the nutritional composition or nutritional supplement of the present disclosure may be in any form that is suitable to administer all the ingredients. For example, the nutritional composition or nutritional supplement of the present disclosure can be in the form of a powdered nutritional composition to be reconstituted in milk or water, a food product, a drink, a nutritional supplement or a nutraceutical.

When the nutritional composition or nutritional supplement of the present disclosure is in the form of a powdered nutritional composition to be reconstituted in milk or water, the nutritional composition or nutritional supplement may preferably comprise a protein source, a carbohydrate source and a lipid source, preferably together with lecithin. The nutritional composition or nutritional supplement may also comprise soya lecithin and/or a bulking agent. The protein source may be dried milk or dried skimmed milk. As carbohydrate source sucrose and/or maltodextrin may be used. The lipid source may be vegetable oil. The formulation may also alternatively or additionally contain glucose syrup, milk fat, magnesium citrate, choline salts and esters, prebiotic fibers, and/or ascorbyl palmitate. Flavor compounds, such as cocoa powder or honey, for example, may be added to provide taste variations.

In another aspect, the nutritional composition or nutritional supplement of the present disclosure may be a product selected from the group consisting of a nutritional product, a functional food product, a healthy ageing product, a dairy product, a dairy alternative product, a beverage product, a diet product, and a pet food product.

### EXAMPLES

### Example 1. Supplement Containing Zinc, Vitamin D and Vitamin B in Preconception and pregnancy increases human milk zinc, vitamin D and vitamin B concentrations

The following non-limiting example presents scientific data developing and supporting the concept of methods and compositions for increasing concentrations of zinc, vitamin D and/or vitamin B in human milk of a subject by using supplements containing zinc, vitamin D and/or vitamin B before or during pregnancy of the subject.

Vitamins and minerals are essential during pregnancy and lactation to support maternal and infant health. For example, zinc is involved in various cellular processes and zinc requirement increases in pregnancy and lactation. Furthermore, vitamin D₃ is involved in glucose homeostasis and bone development in the infants, and B-vitamins are involved in various metabolic processes including energy production.

The aim of this study was to investigate the effects of a nutritional supplement containing zinc and vitamin D₃, starting preconception and taken throughout pregnancy, on concentrations of minerals (zinc, calcium, copper, iodine, iron, magnesium, manganese, phosphorus potassium, selenium and sodium) and vitamins (vitamin D₃ and B-vitamins) in human milk, alongside characterizing longitudinal changes in concentrations of these vitamins.

Human milk samples were collected across 7 time points from 1 week to 12 months from lactating mothers from Singapore (n=158) and New Zealand (n=180). Minerals were quantified using sector field inductively coupled plasma mass spectrometry. Potential intervention effects on human milk mineral concentrations were assessed using linear mixed models with a repeated measures design and time-weighted area-under-the-curve analysis. Vitamin D was quantified using supercritical fluid chromatography, and B-vitamins were quantified using reversed-phase liquid chromatography combined with tandem mass spectrometry. Potential intervention effects on vitamin D₃ and B₁, B₂, B₃, B₆ and B₉ concentrations in human milk were assessed using linear mixed models with a repeated measures design.

Over the first 3 months of lactation, zinc concentrations were higher in the intervention group by 11% compared to the control group (p=0.022). This effect was most evident at 6 weeks of lactation. The intervention did not have any effect on other mineral concentrations in human milk. Longitudinal changes in human milk minerals until 12 months of lactation were also observed. Zinc and copper progressively decreased throughout 12 months while others (iron, potassium, sodium and phosphorus) decreased until 6 months then plateaued. Calcium and magnesium initially increased in early lactation and iodine remained relatively constant throughout 12 months. Manganese and selenium displayed a U-shape pattern during the first 12 months. These contrasting patterns of changes in mineral concentrations during lactation may reflect different critical roles at different stages of infancy. Further studies are required to assess potential health benefits of increased human milk zinc concentrations on infant outcomes, achieved by supplement use.

Over the first 3 months of lactation, intervention group had higher total vitamin D₃ concentrations by 28% (p=0.003), vitamin D₃ concentrations by 46% (p=0.007) and 25-hydroxyvitamin D₃ concentrations by 17% (p=0.002). The intervention effect was most evident in early lactation until 6 weeks. The intervention had no effect on vitamins B₁, B₂, B₃, B₆ and B₉. While total vitamin D₃ concentrations gradually increased over time in the first 12 months of lactation, vitamin B₁, B₂, B₃ and B₆ displayed greatest concentrations in early lactation, around 6 weeks or 3 months. Vitamin B₉ concentrations remained fairly constant during this time. Further studies are required to examine the potential benefits of maternal supplement use on infant health outcomes, mediated by increased human milk vitamin D₃ concentrations.

### Materials and Methods

### Study Design

The detailed study protocol for the NiPPeR study (ClinicalTrials.gov, identifier: NCT02509988, Universal Trial Number U1111-1171-8056; registered on 16 Jul. 2015) has been published previously (Godfrey et al. Trials. 2017; 18(1):1-12). In brief, the NiPPeR study was a double-blind, randomised controlled trial investigating the effects of a nutritional supplement during preconception and pregnancy on maternal pregnancy and infant outcomes. The control supplement comprised of standard amounts of micronutrients that are part of routine pregnancy care (Table 1). In addition to these nutrients, the NiPPeR intervention supplement contained vitamins B₂, B₆, B₁₂, and D, as well as zinc, myo-inositol, and probiotics (Table 1).

The study supplements were packaged as a powder in sachets and were taken twice daily, as a drink reconstituted with water. The study was conducted in Southampton (UK), Singapore, and Auckland (New Zealand), with ethics approval obtained at each site [Southampton—Health Research Authority National Research Ethics Service Committee South Central Research Ethics Committee (15/SC/0142); Singapore—the National Healthcare Group Domain Specific Review Board (2015/00205); and New Zealand—Northern A Health and Disability Ethics Committee (15/NTA/21)]. All participants provided written informed consent.

TABLE 1
Detailed nutrient composition of the intervention and control drinks in the NiPPeR study

| | | | | Daily | | Recommended | |
|---|---|---|---|---|---|---|---|
| Group | Nutrient | Intervention | Control | dose | | range^{#} | |
| Minerals | Calcium | ✓ | ✓ | 150 | mg | 700-1300 | mg |
| | Iodine | ✓ | ✓ | 150 | μg | 140-220 | μg |
| | Iron | ✓ | ✓ | 12 | mg | 14.8-27 | mg |
| | Zinc | ✓ | X | 10 | mg | 7-15 | mg |
| Vitamins | A (β-carotene) | ✓ | ✓ | 720 | μg | 700-750 | μg |
| | B₂ (Riboflavin) | ✓ | X | 1.8 | mg | 1.38-1.46 | mg |
| | B₆ (Pyridoxine) | ✓ | X | 2.6 | mg | 1.2-1.9 | mg |
| | B₉ (Folic acid) | ✓ | ✓ | 400 | μg | 300-600 | μg |
| | B₁₂ (Cobalamin) | ✓ | X | 5.2 | μg | 1.5-2.6 | μg |
| | D₃ (Cholecalciferol) | ✓ | X | 400 | IU (10 μg) | 5-10 | μg |

| | | | | | | |
|---|---|---|---|---|---|---|
| Other | Myo-inositol | ✓ | X | 4 | g | n/a |
| | Lactobacillus rhamnosus * | ✓ | X | >1 × 10⁹ | CFU | n/a |
| | Bifidobacterium animalis ssp. | ✓ | X | >1 × 10⁹ | CFU | n/a |
| | lactis ^{†} | | | | | |

^{#}Recommended ranges for daily intake during pregnancy according to the reference nutrient intake for the UK (HMSO Publications Centre; 1991), recommended dietary allowance for Singapore (National University Hospital. Singapore; 2006), and recommended daily intake for New Zealand (National Health and Medical Research Council; 2006).
* NCC 4007 (CGMCC 1.3724)
^{†} NCC 2818 (CNCM I-3446)
Abbreviations: CFU, colony-forming units; n/a, not applicable.

### Study Participants

Participants were recruited by self-referral after study information was disseminated through local and social media advertisements. The full inclusion, exclusion, and withdrawal criteria have been reported previously (Godfrey et al. Trials. 2017; 18(1):1-12), and are provided in Table 2. Briefly, women aged 18-38 years who were planning to conceive within 12 months were eligible for the study. Eligible participants were randomised to either the control or the intervention group through the electronic study database (Godfrey et al. Trials. 2017; 18(1):1-12), and stratified by site and ethnicity (Caucasian, Chinese, South Asian, Malay and Other) to ensure balanced allocation of participants.

TABLE 2
The inclusion, exclusion, and withdrawal criteria for participants
in the NiPPeR study (adapted from Godfrey et al. 2017).

| Inclusion criteria | Exclusion criteria | Withdrawal criteria |
|---|---|---|
| Women aged 18-38 years | Pregnant or currently | Participant wishes to |
| Living in Southampton, | breastfeeding | withdraw from the study |
| Singapore, or Auckland | Assisted fertility apart | Participant unwilling or |
| In Southampton and | from those taking | unable to comply with the |
| Auckland, planning to have | clomiphene or | study protocol (including |
| future maternity care in | letrozole alone | attendance at study visits, |
| Southampton and Auckland, | Pre-existing type 1 or | undergoing clinical |
| respectively | type 2 diabetes | assessments or biosampling) |
| In Singapore, willing to | (defined as a fasting | An overall uptake level of |
| deliver at the National | plasma glucose | intervention/control |
| University Hospital | concentration ≥7.0 | nutritional drink of less |
| Planning to conceive within | mmol/L or post OGTT | than 60% evidenced by |
| 6 months (although | 2-h plasma glucose | sachet counting, each case |
| conception up to 12 months | concentration ≥11.1 | reviewed by the investigators. |
| after phenotyping deemed | mmol/L) | Pregnancy before or at |
| acceptable) | Oral or implanted | preconception visit 2 |
| In Singapore, being of | contraception | Miscarriage (pregnancy |
| Chinese, Malay, or Indian | currently or in the last | loss before 24 weeks' |
| ethnicity, or of mixed | month, or with an | gestation) or ectopic |
| Chinese/Malay/Indian | intrauterine | pregnancy. In the event of |
| ethnicity | contraceptive device in | a first-trimester pregnancy |
| Ability to provide written | situ | loss, participant could re- |
| informed consent | Use of metformin or | join the study with the |
| | systemic steroids | same randomisation code |
| | currently or in the last | Multiple pregnancy (twins |
| | month | or other multiples) |
| | Use of anticonvulsant | Infant death in the |
| | medication currently | perinatal period (for post- |
| | or in the last month | birth secondary outcomes) |
| | Treatment for HIV, | Any adverse reaction |
| | Hepatitis B or C | deemed by the |
| | currently or in the last | investigators to be causally |
| | month | related to the intervention |
| | Known serious food | Withdrawal at the |
| | allergy | discretion of the |
| | | investigators for medical |
| | | reasons |

### Human Milk Sample Collection

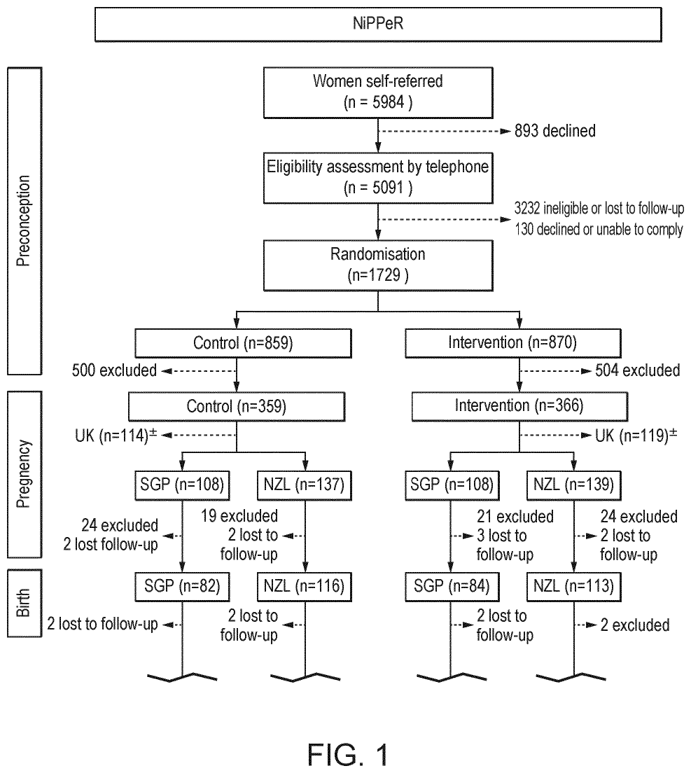

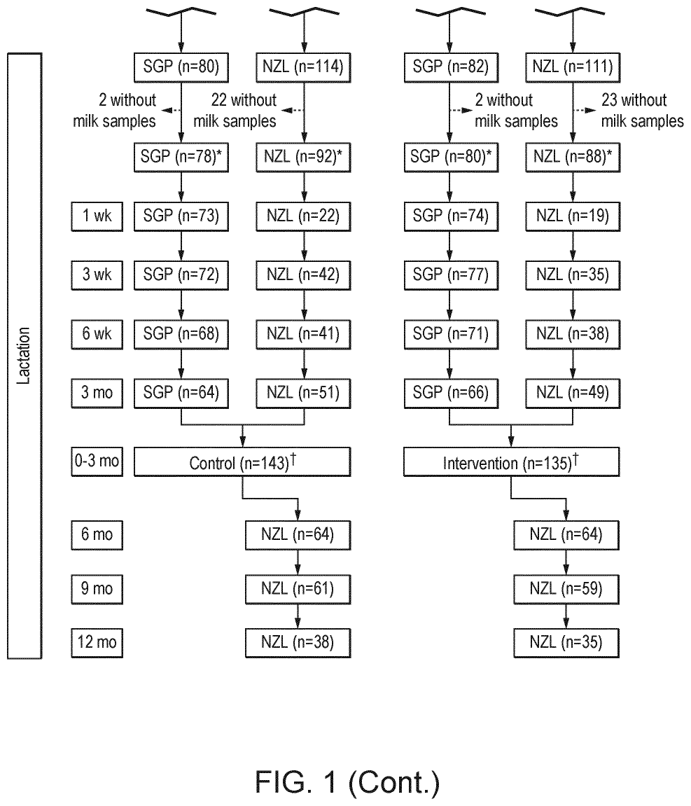

HM samples were collected only in Singapore (from July 2016) and New Zealand (from May 2017) (FIG. 1). Samples were collected during clinic visits at 1 week+3 days, 3 weeks+5 days, 6 weeks+5 days, and 3 months+10 days (4 time points); in New Zealand, there were additional HM collections at 6 months+14 days, 9 months+14 days, and 12 months+14 days (7 time points overall). In Singapore, samples could only be collected until 3 months due to logistical constraints. HM samples were collected in the morning, and mothers were asked to refrain from breastfeeding for 2 hours prior to collection on the side of the breast from where samples would be collected. Whole HM samples were collected from a single breast using an Ameda Lactaline breast pump (Ameda, Inc, Murarrie, Australia) (Samuel et al. Front Nutr. 2020; 7 (September)). The breast was pumped for 15 minutes or until fully emptied, under the supervision of a breastfeeding counsellor. Soon after collection, HM samples were vortexed for homogenization and then stored at −80° C. until analysis. HM samples were not collected if the mother had ceased breastfeeding, her milk supply was low, or there were complications with milk expression. The total number of samples collected at each time point are outlined in FIG. 1. The number of participants with longitudinal samples during the first 3 months of lactation is summarised in Table 3, and during the first 12 months of lactation in New Zealand in Table 4.

TABLE 3
Number of longitudinal human milk (HM) samples collected
per participant in the first 3 months of lactation
in Singapore (SGP) and New Zealand (NZL).

| | Control | | | Intervention | | |
|---|---|---|---|---|---|---|
| HM samples (n) | Overall | SGP | NZL | Overall | SGP | NZL |
| 0 | 27 | — | 27 | 33 | — | 33 |
| 1 | 22 | 3 | 19 | 16 | 2 | 14 |
| 2 | 25 | 9 | 16 | 18 | 7 | 11 |
| 3 | 23 | 8 | 15 | 27 | 12 | 15 |
| 4 | 73 | 58 | 15 | 74 | 59 | 15 |
| Total | 170 | 78 | 92 | 168 | 80 | 88 |

TABLE 4
Number of longitudinal human milk (HM) samples collected
in New Zealand during the first 12 months of lactation.

| HM samples (n) | Control | Intervention |
|---|---|---|
| 1 | 21 | 16 |
| 2 | 17 | 17 |
| 3 | 10 | 20 |
| 4 | 13 | 7 |
| 5 | 11 | 12 |
| 6 | 13 | 11 |
| 7 | 7 | 5 |
| Total | 92 | 88 |

### Human Milk Mineral Quantification

HM mineral quantification was carried out by ALS Scandinavia AB (Luleå, Sweden). HM calcium, cobalt, copper, iron, potassium, magnesium, manganese, sodium, nickel, phosphorus, selenium, and zinc were quantified using Sector Field Inductively Coupled Plasma Mass Spectrometry (SF-ICP-MS), ELEMENT 2 (Thermo, Bremen, Germany) equipped with an ASX 500 sample changer (CETAC Technologies Inc., Omaha, USA), as described by Rodushkin et al. (Rodushkin et al., Fresenius J Anal Chem. 1999; 364(4):338-46; J Anal At Spectrom. 2000; 15(8):937-44). Their method of sample preparation was modified slightly by reducing the sample intake for the microwave-assisted acidic decomposition to 0.2 mL. Briefly, HM samples (0.2 mL), were transferred in perfluoroalkoxy polymer lined vessels and mineralized with a microwave oven (MDS-2000, CEM Corporation, Matthews, USA) using analytical grade nitric acid (Merck, Darmstadt, Germany) after additional purification by sub-boiling distillation in a quartz still. After mineralization, the resulting solutions were diluted with Milli-Q water (Millipore Milli-Q, Bedford, USA) and spiked with internal standard solution containing scandium, indium, and lutetium. Iodine was also quantified using SF-ICP-MS ELEMENT 2 with an ASX 500 sample changer, but following the instrumental method by Engstrom et al. (Anal Chim Acta. 2004; 521(2):123-35). Sample preparation was slightly modified from Krachler et al. (J Anal At Spectrom. 2009; 24(5):605-10) using the alkaline reagent composition given by Engstrom et al. (Anal Chim Acta. 2004; 521(2):123-35) and a dilution factor of 1:50. Briefly, prior to ICP-MS analysis, HM samples were diluted (with a dilution factor of 1:50) with alkaline diluent containing 0.01 M ammonia (Suprapur, Merck), 0.2 mM (NH₄)₂EDTA (Fluka) and 0.07% Triton X-100 (Merck). All intra- and inter-assay coefficients of variation were <10%.

### Statistical Analyses

Mineral concentration measurements below the lower level of quantification (LLoQ) were assigned a value of 0.5×LLoQ (Table 5). To minimize the removal of values from the data set, we adopted a highly conservative approach defining extreme values (i.e., outliers) as measurements outside the mean±5 standard deviations (SD) range. There were no values below this range, but for some minerals there were a few values greater than the mean+5 SD classified as extreme (i.e., >99.99997^{th} percentile), ranging from nil to 1.01% of their respective samples (Table 10), and removed from analyses. Further, it was not possible to undertake reliable statistical analyses on cobalt and nickel as a large proportion of values were below the LLoQ (41.4% and 83.0%, respectively). For all other minerals, data were log-transformed to approximate a normal distribution, then back-transformed for reporting.

TABLE 5
Number of values for each mineral measured in human milk in the
NiPPeR study, whose concentrations were below the lower limit
of quantification (LLoQ) or classified as extreme values.

| Mineral | <LLoQ | <Mean − 5 SD | >Mean + 5 SD |
|---|---|---|---|
| Zinc | nil | nil | nil |
| Calcium | nil | nil | nil |
| Cobalt | 490 (41.4%) | n/a | n/a |
| Copper | nil | nil | 3 (0.25%) |
| Iodine | nil | nil | 6 (0.50%) |
| Iron | nil | nil | 8 (0.68%) |
| Magnesium | nil | nil | nil |
| Manganese | 2 (0.17%) | nil | 7 (0.59%) |
| Nickel | 983 (83.0%) | | |
| Phosphorus | nil | nil | 1 (0.08%) |
| Potassium | nil | nil | nil |
| Selenium | nil | nil | 5 (0.42%) |
| Sodium | nil | nil | 12 (1.01%) |

n/a, not applicable; SD, standard deviation.

Potential intervention effects on HIM mineral concentrations were only examined on the samples collected in the first 3 months of lactation, which were collected in both Singapore and New Zealand. These were assessed using linear mixed models with a repeated measures design. Parameters included were randomization group, visit, their interaction term (group*visit), and study site, as well as maternal pre-pregnancy body mass index (BMI) and adherence to the study protocol as covariates. The participant's study ID was also included as a random factor to account for the multiple measurements on the same individual (non-independence). If the interaction term was statistically significant, between-group comparisons were only reported on a per-visit basis.

The time-weighted area-under-the-curve (TwAUC) was also calculated for each participant who had at least 3 valid HIM measurements within the first 3 months of lactation, using the following formula:

$$TwAUC = \frac{AUC}{{age}_{t} - {age}_{0}}$$

Where age₀ and ageₜ were the infant's ages when the first and last measurements used in the AUC were collected, respectively. TwAUC data were analysed using general linear models adjusted for study site, maternal BMI, and adherence.

Subgroup analyses were also performed to examine potential treatment effects over the first 3 months of lactation separately for Singapore and New Zealand. Temporal changes in HIM minerals from 1 week to 12 months of lactation were plotted and reported for the New Zealand site only.

Study outcomes are reported as the back-transformed least-squares means (i.e. adjusted means) for each group or the adjusted mean differences (aMD) between groups, and their respective 95% confidence intervals (CI). Note that the aMD for back-transformed values represent proportional differences between groups. Statistical analyses were carried using SAS version 9.4 (SAS Institute Inc., Cary, NC, USA) and graphs created with GraphPad Prism version 8.2.1 (GraphPad Software, San Diego, California USA). All statistical tests were two-sided with significance maintained at p<0.05, without adjustments for multiple comparisons or imputation of missing values.

### Human Milk Vitamin D Quantification

Quantification of vitamin D in HM was undertaken as described previously (Oberson J M, Bénet S, Redeuil K, Campos-Gimenez E. Quantitative analysis of vitamin D and its main metabolites in human milk by supercritical fluid chromatography coupled to tandem mass spectrometry. Anal Bioanal Chem. 2020; 412(2):365-75). HIM aliquots were thawed by placing the tubes at 40° C. for one hour. After vigorous vortexing to ensure homogenisation, a 200 μL portion of each tube were transferred into a 15 mL polypropylene tube. Up to 5 or 6 aliquots were then pooled for a total volume of 1 mL. Then, 1 mL of ethanol was added, the tubes were vortexed and stored at −20° C. until further extraction steps.

To the ethanolic extracts, 25 μL of internal standard solution and 2.5 mL of hexane:ethyl acetate (90:10) were added, the tubes were tightly closed and shaken for 180 seconds in a Geno/Grinder©. The tubes were then centrifuged at 2,500×g at room temperature and the upper phase was carefully transferred to an 8 mL glass tube. The liquid-liquid extraction was repeated, and the upper phases were combined. The pooled upper phases were evaporated to dryness under a stream of nitrogen gas at room temperature. The dried residue was transferred quantitatively into a 1.5 mL microcentrifuge tube using 3 portions of 400 μL of hexane-ethyl acetate (90:10), vortexing and pooling the hexane-ethyl acetate portions pooled into a microcentrifuge tube. After evaporation to dryness, 1 mL of isooctane and 100 mg of sodium sulfate were added, the tubes were centrifuged and the supernatant was transferred to a microcentrifuge tube for PTAD derivatization. 25 μL of PTAD solution were added, the tubes were vortexed and let stand at room temperature for one hour, protected from light. [00135]100 μL of acetonitrile:water (90:10) was added to quench the derivatization reaction and extract vitamin D-PTAD derivatives. After vortexing and centrifuging the tubes, about 50 μL of lower phase were transferred to a 2 mL amber glass vial and was analysed by SFC-MS/MS.

Calibration curves were created with each series of analysis (20 samples). Two QC samples (low and high) were created by spiking a pooled HM (naturally containing vitamin D₃ and 25(OH)D₃) to yield about 20 ng/100 mL and about 40 ng/100 mL concentrations of the metabolites, respectively. After spiking about 200 mL of milk, the QCs were aliquoted into 1 mL tubes and at −20 degrees C. Analytical batches included the 2 QC samples extracted in duplicate and were run from June 2018 to November 2020. Relative standard deviation of repeatability (RSDr) and of intermediate reproducibility (RSDiR) were calculated according to ISO 5727 from the data (n=42) and warning and control limits was defined at 95% and 99% confidence levels. RSDr varied between 9.9% and 14.4% on all four analytes, while RSDiR were between 14.9% and 25.9%. Although the values were higher than the ones obtained during method validation, they better represent the performance of the method on a large number of replicates and during a longer period of time (2 years). No trend towards degradation was observed on any of the compounds during the 2 years of analysis, demonstrating the stability of the control samples.

Lower and upper limits of quantification (LLoQ and ULoQ, respectively) were defined as the lowest point of calibration (0.02 ng/100 μL) and the highest concentration of standard for which linearity has been proven for (100 ng/100 μL), corresponding to 2 ng/100 mL and 10,000 ng/100 mL when expressed in the sample considering dilution factors.

All results were reported as mass unit (ng/100 mL) for each of the metabolites (vitamin D₃, 25(OH)D₃, vitamin D₂, 25(OH)D₂).

### Human Milk Vitamin B Quantification

Quantification of B-vitamins in HM were undertaken as described previously (Redeuil K, Bénet S, Affolter M, Thakkar K S, Campos Gimenez E. A Novel Methodology for the Quantification of B-Vitamers in Breast Milk. J Anal Bioanal Tech. 2017; 08(02):1-10; and Redeuil K, Vulcano J, Prencipe F P, Bénet S, Campos-Gimenez E, Meschiari M. First quantification of nicotinamide riboside with B3 vitamers and coenzymes secreted in human milk by liquid chromatography-tandem-mass spectrometry. J Chromatogr B. 2019; 74-80). Briefly, 200 μL of HM were exposed to methanolic protein precipitation. After evaporation, reconstitution and filtration, sample extracts were analyzed by reversed-phase liquid chromatography combined with tandem mass spectrometry. The content of each vitamer (individual molecule) was express individually. In addition, the content of each of the vitamins was calculated as the sum of all the defined contributing vitamers to a vitamin activity as detailed in Table 6.

TABLE 6
List of vitamin groups and their vitamer constituents.

| Vitamin Group | Vitamers |
|---|---|
| Total vitamin D₃ | Vitamin D₃ |
| | 25(OH)D₃ |
| Vitamin B₁ | Thiamine |
| | TMP |
| | TPP |
| Vitamin B₂ | FAD |
| | FMN |
| | Riboflavin |
| Vitamin B₃ | Nicotinamide |
| | NMN |
| | NRT |
| | Nicotinic Acid |
| Vitamin B₆ | Pyridoxal |
| | PLP |
| | Pyridoxamine |
| | PMP |
| | Pyridoxine |
| Vitamin B₉ | Folic Acid |
| | 5MeTHF |
| Individual vitamers | NAD |
| | NADP |

Abbreviations: 25(OH)D₃, 25-hydroxyvitamin D₃; 5MeTHF, 5-methyl tetrahydrofolic acid; FAD, flavin adenine dinucleotide; FMN, flavin mononucleotide; NAD, nicotinamide adenine dinucleotide; NAPD, nicotinamide adenine dinucleotide phosphate; NMN, nicotinamide mononucleotide; NRT, nicotinamide riboside; PLP, pyridoxal 5′-phosphate; PMP, pyridoxamine-5′-phsophate; TMP, thiamine monophosphate; TPP, thiamine pyrophosphate.

### Statistical Analysis

Vitamer concentration measures below the LLoQ were assigned a value of 0.5×LLoQ (Table 7). To minimise the removal of values from the dataset, we adopted a conservative approach defining extreme values (i.e., outliers) as measurements outside the mean±standard deviations (SD) range. There were no values below mean−5SD, but for some vitamers there were a few values greater than the mean+5 SD classified as extreme values (i.e., >99,99997^{th} percentile) (Table 7), and removed from analyses. Further, it was not possible to undertake reliable statistical analysis on some vitamers (vitamin D₂, 25(OH)D₂, nicotinic acid, pyridoxamine, pyridoxine, and folic acid) as a large portion of their values (>50%) were below the LLoQ. For all other vitamers, data were log-transformed to approximate a normal distribution, then back-transformed for reporting.

TABLE 7
Number of values for each vitamin variable measured in
the NiPPeR study, whose concentrations were below the
lower limit of quantification (LLoQ), above the upper
limit of quantification (ULoQ) or classified as values.

| Vitamin | <LLoQ | >ULoQ | >Mean + 5 SD |
|---|---|---|---|
| Total Vitamin D₃ | n/a | n/a | 21 (1.9%) |
| Vitamin D₃ | 139 (12.2%) | n/a | 12 (1.1%) |
| 25(OH)D₃ | 6 (0.53%) | n/a | 5 (0.44%) |
| Vitamin D₂ | 864 (75.7%) | n/a | n/a |
| 25(OH)D₂ | 970 (85.0%) | n/a | n/a |
| Vitamin B1 | n/a | n/a | 1 (0.08%) |
| Thiamine | 107 (9.0%) | nil | 1 (0.08%) |
| TMP | 27 (2.3%) | nil | 2 (0.17%) |
| TPP* | 49 (4.1%) | nil | 6 (0.51%) |
| Vitamin B2 | n/a | n/a | 3 (0.25%) |
| FAD | Nil | nil | 2 (0.17%) |
| FMN | 11 (0.93%) | 1 (0.08%) | 2 (0.17%) |
| Riboflavin | 30 (2.5%) | 11 (0.93%) | 15 (1.27%) |
| Vitamin B3 | n/a | n/a | nil |
| Nicotinamide | Nil | nil | 2 (0.17%) |
| NMN | 13 (1.1%) | nil | 1 (0.08%) |
| NRT | 103 (8.7%) | 4 (0.34%) | nil |
| Nicotinic acid | 853 (72.1%) | nil | 7 (0.59%) |
| NAD | nil | 10 (0.84%) | nil |
| NADP | 1 (0.08%) | 2 (0.17%) | 4 (0.34%) |
| Vitamin B6 | n/a | n/a | 4 (0.34%) |
| Pyridoxal | 39 (3.3%) | nil | 8 (0.68%) |
| PLP | 74 (6.3%) | nil | 4 (0.34%) |
| Pyridoxamine | 612 (51.7%) | nil | 7 (0.59%) |
| PMP | 189 (16.0%) | nil | 3 (0.25%) |
| Pyridoxine | 925 (78.2%) | 1 (0.08%) | 10 (0.85%) |
| Vitamin B9 | n/a | n/a | 1 (0.08%) |
| Folic Acid | 515 (43.5%) | nil | 5 (0.42%) |
| 5MeTHF | nil | 1 (0.08%) | 5 (0.42%) |

n/a, not applicable; SD, standard deviation.
*78 values of TPP could nt be accepted due to analytical issues, these values were considered as missing.
Abbreviations: 25(OH)D₃, 25-hydroxyvitamin D₃; 5MeTHF, 5-methyl tetrahydrofolic acid; FAD, flavin adenine dinucleotide; FMN, flavin mononucleotide; NAD, nicotinamide adenine dinucleotide; NAPD, nicotinamide adenine dinucleotide phosphate; NMN, nicotinamide mononucleotide; NRT, nicotinamide riboside; PLP, pyridoxal 5′-phosphate; PMP, pyridoxamine-5′-phsophate; TMP, thiamine monophosphate; TPP, thiamine pyrophosphate.

Vitamers belonging to the same vitamin group were summed together to give the total HM vitamin concentrations (Table 6). At a given visit, the total vitamin concentration value was not included in the analysis if at least one of its constituting vitamer value was greater than the mean+5 SD threshold. Further, vitamin values greater than the mean+5 SD were classified as extreme and removed from analyses. For all vitamins, data were log-transformed to approximate a normal distribution, then back-transformed for reporting.

Potential intervention effects on HM vitamin concentrations were only examined on the samples collected in the first 3 months of lactation, which were collected in both Singapore and New Zealand. In a sensitivity analysis, this was also assessed in a subgroup of participants who provided consecutive samples across the 4 time points in the first 3 months. These were assessed using linear mixed models with a repeated measures design. Key parameters included were randomisation group, visit, their interaction term (group*visit), and study site, maternal pre-pregnancy body mass index and adherence to the study protocol. For vitamin D, covariates that were also included were maternal serum preconception 25(OH)D₃ levels and season at the time of HM collection. For vitamin B, infant gestational age was also included as a covariate. The participant's study ID was also included as a random factor to account for the multiple measurements on the same individual (non-independence). If the interaction term was statistically significant, between-group comparisons were only reported on a per-visit basis.

Subgroup analysis were also performed to examine potential treatment effects over the first 3 months of lactation separately for Singapore and New Zealand. Temporal changes in HM vitamins from 1 week to 12 months of lactation were plotted and reported for the New Zealand site only. This was also examined in a subgroup of New Zealand participants who provided HM samples for at least five out of six time points between 3 weeks and 12 months.

Study outcomes are reported as the back-transformed least-square means (i.e., adjusted means) for each group or the adjusted mean difference (aMD) between groups, and their respective 95% confidence intervals (CI). Note that the aMD for back-transformed values represent proportional differences between intervention and control groups. Statistical analysis was carried out using R version 4.1.0 (R Foundation for Statistical Computing, Vienna, Austria), and SAS version 9.4 (SAS Institute Inc., Cary, NC, USA). Graphs were created with GraphPad Prism version 8.2.1 (GraphPad Software, San Diego, California USA). All statistical tests were two-sided with significance maintained at p<0.05, without adjustments for multiple comparisons or imputation of missing values.

### Results

### Study Population

Maternal demographic and pre-pregnancy BMI characteristics were similar in control and intervention groups (Table 8), noting that participants were mostly Chinese in Singapore and Caucasian in New Zealand (Table 9). Adherence to the study drinks was high, the average of the total cohort being 87.2%. Perinatal outcomes were also similar between the two groups overall (Table 8) and within sites (Table 9).

TABLE 8
Baseline and perinatal characteristics of participants
in the NiPPeR study who provided at least one human
milk sample in the first 12 months of lactation.

| | Overall (n = 338) | |
|---|---|---|
| | Control | Intervention |
| n | 170 (50.3%) | 168 (49.7%) |
| Ethnicity | | |
| Caucasian | 70 (41.2%) | 67 (39.9%) |
| Chinese | 70 (41.2%) | 69 (41.1%) |
| South Asian | 10 (5.9%) | 10 (6.0%) |
| Malay | 10 (5.9%) | 10 (6.0%) |
| Other | 10 (5.9%) | 12 (7.1%) |
| Adherence (%) | 87.4 ± 11.2 | 86.9 ± 13.4 |
| Age at delivery (years) | 31.9 ± 2.9 | 32.4 ± 3.2 |
| BMI (kg/m²) | 24.4 ± 5.2 | 23.4 ± 4.4 |
| BMI status | | |
| Underweight or normal weight | 100 (58.8%) | 103 (61.3%) |
| Overweight | 41 (24.1%) | 48 (28.6%) |
| Obesity | 29 (17.1%) | 16 (9.5%) |
| Highest level of education | | |
| Bachelor′s degree or higher | 137 (80.6%) | 136 (81.0%) |
| Lesser qualification* | 33 (19.4%) | 32 (19.0%) |
| Household income quintile | | |
| 5 (lowest) | 4 (2.4%) | 1 (0.6%) |
| 4 | 12 (7.1%) | 16 (9.5%) |
| 3 | 44 (25.9%) | 43 (25.6%) |
| 2 | 60 (35.3%) | 55 (32.7%) |
| 1 (highest) | 44 (25.9%) | 43 (25.6%) |
| Missing | 6 (3.5%) | 10 (6.0%) |
| Smoking during pregnancy | | |
| None | 134 (78.8%) | 148 (88.6%) |
| Passive | 33 (19.4%) | 16 (9.6%) |
| Active | 3 (1.8%) | 3 (1.8%) |
| Alcohol consumption (units per week) | | |
| None | 45 (26.5%) | 44 (26.2%) |
| ≤2.5 | 73 (42.9%) | 68 (40.5%) |
| >2.5 and ≤7 | 30 (17.6%) | 23 (13.7%) |
| >7 and ≤14 | 17 (10.0%) | 23 (13.7%) |
| >14 | 5 (2.9%) | 10 (6.0%) |
| GDM | | |
| No GDM | 126 (74.1%) | 125 (74.4%) |
| GDM | 42 (24.7%) | 43 (25.6%) |
| Missing | 2 (1.2%) | — |
| Mode of delivery | | |
| Vaginal delivery | 125 (73.5%) | 119 (70.8%) |
| Caesarean section | 44 (25.9%) | 49 (29.2%) |
| Gestational age (weeks) | 39.1 ± 1.6 | 39.2 ± 1.5 |
| Preterm | 14 (8.2%) | 11 (6.5%) |
| Term or post-term | 156 (91.8%) | 157 (93.5%) |
| Parity | | |
| Primiparous | 114 (67.1%) | 95 (56.5%) |
| Multiparous | 56 (32.9%) | 73 (43.5%) |
| Infant sex | | |
| Male | 76 (44.7%) | 79 (47.0%) |
| Female | 94 (55.3%) | 89 (53.0%) |

Data are n (%), or mean ± standard deviation (SD). Adherence to the study protocol was determined by sachet counting. Body mass index (BMI) status was defined using ethnic-specific thresholds for BMI categories: for Asians, under or normal weight <23.0 kg/m², overweight 23.0-27.49 kg/m², obesity ≥27.5 kg/m²; for non-Asians, under or normal weight <25.0 kg/m², overweight 25.0-29.99 kg/m², obesity ≥30.0 kg/m². Alcohol consumption was collected at the first pre-conception visit as weekly alcohol consumption in the last 3 months. Gestational diabetes (GDM) was defined by International Association of Diabetes and Pregnancy Study Groups criteria (Diabetes Care. 2010; 33(3): 676-82). Gestational age was determined using a pre-specified algorithm as previously described (Thorax. 2010; 65(12): 1099-106) with preterm defined as birth <37 weeks of gestation, and term or post-term as birth at ≥37 weeks of gestation. Breastfeeding duration in weeks determined by age last fed direct or expressed human milk.
*Including incomplete and complete high school qualifications, and other tertiary level qualifications below bachelors (e.g. diploma or certificate).

TABLE 9
Baseline and perinatal characteristics of participants in the NiPPeR study by site.

| | Singapore (n = 158) | | New Zealand (n = 180) | |
|---|---|---|---|---|
| | Control | Intervention | Control | Intervention |
| n | 78 (49.4%) | 80 (50.6%) | 92 (51.1%) | 88 (48.9%) |
| Ethnicity | | | | |
| Caucasian | — | — | 70 (76.1%) | 67 (76.1%) |
| Chinese | 62 (79.5%) | 62 (77.5%) | 8 (8.7%) | 7 (8.0%) |
| South Asian | 5 (6.4%) | 7 (8.8%) | 5 (5.4%) | 3 (3.4%) |
| Malay | 10 (12.8%) | 10 (12.5%) | — | — |
| Other | 1 (1.3%) | 1 (1.3%) | 9 (9.8%) | 11 (12.5%) |
| Adherence (%) | 87.2 ± 12.4 | 85.5 ± 14.9 | 87.7 ± 10.2 | 88.2 ± 11.9 |
| Age at delivery(years) | 32.7 ± 3.3 | 31.6 ± 2.8 | 32.2 ± 3.0 | 32.7 ± 3.3 |
| BMI (kg/m²) | 24.5 ± 4.9 | 23.5 ± 5.3 | 25.1 ± 5.0 | 24.5 ± 4.9 |
| BMI status | | | | |
| Underweight or Normal weight | 45 (57.7%) | 46 (57.5%) | 55 (59.8%) | 57 (64.8%) |
| Overweight | 18 (23.1%) | 29 (36.3%) | 23 (25.0%) | 19 (21.6%) |
| Obesity | 15 (19.2%) | 4 (5.0%) | 14 (15.2%) | 12 (13.6%) |
| Highest level of education | | | | |
| Bachelor′s degree or higher | 61 (78.2%) | 64 (80.0%) | 76 (82.6%) | 72 (81.8%) |
| Lesser qualification* | 17 (21.8%) | 16 (20.0%) | 16 (17.4%) | 16 (18.2%) |
| Household income quintile | | | | |
| 5 (lowest) | 3 (3.8%) | 1 (1.3%) | 1 (1.1%) | — |
| 4 | 10 (12.8%) | 13 (16.3%) | 2 (2.2%) | 3 (3.4%) |
| 3 | 25 (32.1%) | 28 (35.0%) | 19 (20.7%) | 15 (17.0%) |
| 2 | 24 (30.8%) | 24 (30.0%) | 36 (39.1%) | 31 (35.2%) |
| 1 (highest) | 13 (16.7%) | 11 (13.8%) | 31 (33.7%) | 32 (36.4%) |
| Missing | 3 (3.8%) | 3 (3.8%) | 3 (3.3%) | 7 (8.0%) |
| Smoking during pregnancy | | | | |
| None | 58 (74.4%) | 66 (82.5%) | 76 (82.6%) | 82 (94.3%) |
| Passive | 19 (24.4%) | 11 (13.8%) | 14 (15.2%) | 5 (5.7%) |
| Active | 1 (1.3%) | 3 (3.8%) | 2 (2.2%) | — |
| Alcohol consumption (units per week) | | | | |
| None | 35 (44.9%) | 33 (41.3%) | 10 (10.9%) | 11 (12.5%) |
| ≤2.5 units | 36 (46.2%) | 42 (52.5%) | 37 (40.2%) | 26 (29.5%) |
| >2.5 and ≤7 | 7 (9.0%) | 4 (5.0%) | 23 (25.0%) | 19 (21.6%) |
| units | | | | |
| >7 and ≤14 | — | 1 (1.3%) | 17 (18.5%) | 22 (25.0%) |
| units | | | | |
| >14 units | — | — | 5 (5.4%) | 10 (11.4%) |
| GDM | | | | |
| No GDM | 52 (66.7%) | 51 (63.7%) | 74 (80.4%) | 74 (84.1%) |
| GDM | 26 (33.3%) | 29 (36.3%) | 16 (17.4%) | 14 (15.9%) |
| Missing | — | — | 2 (2.2%) | — |
| Mode of Delivery | | | | |
| Vaginal delivery | 63 (80.8%) | 60 (75.0%) | 62 (67.4%) | 59 (67.0%) |
| Caesarean section | 14 (17.9%) | 20 (25.0%) | 30 (32.6%) | 29 (33.0%) |
| Gestational age (weeks) | 39.7 ± 1.4 | 38.8 ± 1.5 | 39.5 ± 1.6 | 39.7 ± 1.4 |
| Preterm | 6 (7.7%) | 8 (10.0%) | 8 (8.7%) | 3 (3.4%) |
| Term or Post-term | 72 (92.3%) | 72 (90.0%) | 84 (91.3%) | 85 (96.6%) |
| Parity | | | | |
| Primiparous | 48 (61.5%) | 35 (43.8%) | 66 (71.7%) | 60 (68.2%) |
| Multiparous | 30 (38.5%) | 45 (56.3%) | 26 (28.3%) | 28 (31.8%) |
| Infant sex | | | | |
| Male | 41 (52.6%) | 37 (46.3%) | 35 (38.0%) | 42 (47.7%) |
| Female | 37 (47.4%) | 43 (53.8%) | 57 (62.0%) | 46 (52.3%) |

Data are n (%), or mean ± standard deviation (SD). Adherence to the study protocol was determined by sachet counting. Body mass index (BMI) status was defined using ethnic-specific thresholds for BMI categories: for Asians, under or normal weight <23.0 kg/m², overweight 23.0-27.49 kg/m², obesity ≥27.5 kg/m²; for non-Asians, under or normal weight <25.0 kg/m², overweight 25.0-29.99 kg/m², obesity ≥30.0 kg/m². Alcohol consumption was collected at the first pre-conception visit as weekly alcohol consumption in the last 3 months. Gestational diabetes (GDM) was defined by International Association of Diabetes and Pregnancy Study Groups criteria (Diabetes Care. 2010; 33(3): 676-82). Gestational age was determined using a pre-specified algorithm as previously described (Thorax. 2010; 65(12): 1099-106) with preterm defined as birth <37 weeks of gestation, and term or post-term as birth at ≥37 weeks of gestation. Breastfeeding duration in weeks determined by age last fed direct or expressed human milk.
*Including incomplete and complete high school qualifications, and other tertiary level qualifications below bachelors (e.g. diploma or certificate).

### IMPACT of Intervention on Zinc and Other Minerals

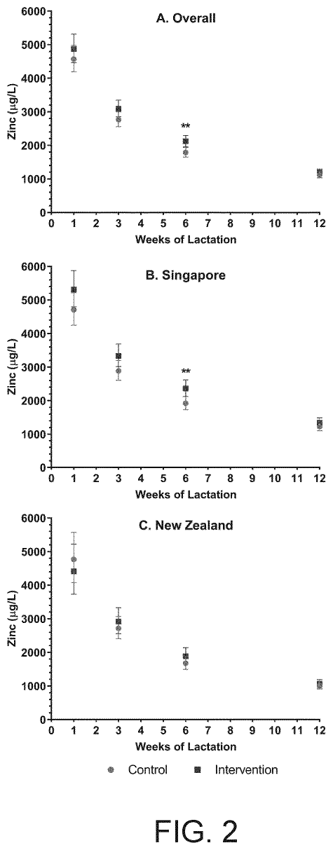

HM zinc concentrations over the first 3 months of lactation were 11% higher in the intervention than in the control group (p=0.022; Table 10), with a similar difference observed for the TwAUC (p=0.021; Table 11). When zinc concentrations at individual visits were examined, the difference between groups was more evident at 6 weeks: 2122 (2299, 1958) and 1789 (1936, 1652) μg/L in the intervention and control group, respectively (p=0.003; FIG. 2A).

TABLE 10
Comparisons in average mineral concentrations in
human milk (HM) over the first 3 months of lactation
in the intervention and control groups.

| Mineral | Intervention | Control | aMD | p-value |
|---|---|---|---|---|
| Zn (μg/L)* | 2485 (2334, | 2244 (2110, | 1.107 (1.015, | 0.022 |
| | 2646) | 2387) | 1.208) | |
| Calcium | 286 (277, | 287 (278, | 0.996 (0.951, | 0.868 |
| (mg/L)^{†} | 296) | 297) | 1.043) | |
| Copper (μg/L) | 386 (373, | 387 (374, | 0.998 (0.950, | 0.922 |
| | 400) | 401) | 1.048) | |
| Iodine (μg/L)^{†} | 113 (105, | 117 (109, | 0.970 (0.881, | 0.530 |
| | 121) | 125) | 1.068) | |
| Iron (mg/L)^{†} | 0.25 (0.24, | 0.24 (0.23, | 1.027 (0.951, | 0.496 |
| | 0.27) | 0.26) | 1.109) | |
| Magnesium | 29.0 (28.3, | 28.5 (27.8, | 1.017 (0.980, | 0.366 |
| (mg/L) | 29.8) | 29.3) | 1.056) | |
| Manganese | 2.55 (2.39, | 2.42 (2.27, | 1.054 (0.964, | 0.249 |
| (μg/L) | 2.72) | 2.58) | 1.152) | |
| Phosphorus | 153 (148, | 150 (145, | 1.020 (0.978, | 0.379 |
| (mg/L) | 158) | 155) | 1.067) | |
| Potassium | 544 (534, | 548 (538, | 0.993 (0.968, | 0.573 |
| (mg/L) | 554) | 558) | 1.018) | |
| Selenium | 16.5 (16.0, | 16.6 (16.2, | 0.990 (0.951, | 0.610 |
| (μg/L) | 16.9) | 17.1) | 1.030) | |
| Sodium | 143 (135, | 148 (140, | 0.963 (0.887, | 0.372 |
| (mg/L) | 152) | 157) | 1.046) | |

*Mineral present only in the intervention drink.
^{†}Minerals present in both control and intervention drinks. Data are the least-squares mean (i.e. adjusted mean) for each group or the adjusted mean difference (aMD) and respective 95% confidence intervals derived from repeated measures analyses, adjusted for visit, an interaction term (group*visit), study site, adherence, and maternal pre-pregnancy body mass index. All data have been log-transformed to approximate a normal distribution, and then back-transformed, so the aMD represents a proportional difference between groups (i.e. intervention vs control). Bold font indicates a statistically significant difference between groups (at p < 0.05).

TABLE 11
Time-weighted area-under-the-curve for zinc concentrations (μg/mL/day) in human
milk samples collected in the NiPPeR study between birth and 3 months of age.

| | Sample size | | | | |
|---|---|---|---|---|---|
| | (Intervention/ | | | | p- |
| Site | Control) | Intervention | Control | aMD | value |
| Overall | 100/96 | 2400 (2254, 2546) | 2163 (2014, 2313) | 236 (36, 437) | 0.021 |
| Singapore | 70/66 | 2752 (2581, 2923) | 2475 (2300, 2652) | 276 (29, 524) | 0.029 |
| New Zealand | 30/30 | 2061 (1818, 2304) | 1878 (1635, 2121) | 183 (−161, 527) | 0.292 |

Data are the least-squares means (i.e. adjusted means) for each group or the adjusted mean differences (aMD) and respective 95% confidence intervals, adjusted for group, visit, study site, adherence, and maternal pre-pregnancy body mass index. Bold font indicates a statistically significant difference between groups (at p < 0.05).

In Singapore, average zinc concentrations over the first 3 months were 15% higher in the intervention group compared to controls (p=0.016; Table 5), as also observed for the TwAUC (p=0.029; Table 6). This difference was also more evident at 6 weeks [2362 (2619, 2132) vs 1921 (2132, 1730) μg/L, respectively; p=0.006](FIG. 2B). No differences in zinc concentrations were detected in New Zealand, although the overall pattern was also observed at that site (FIG. 2C).

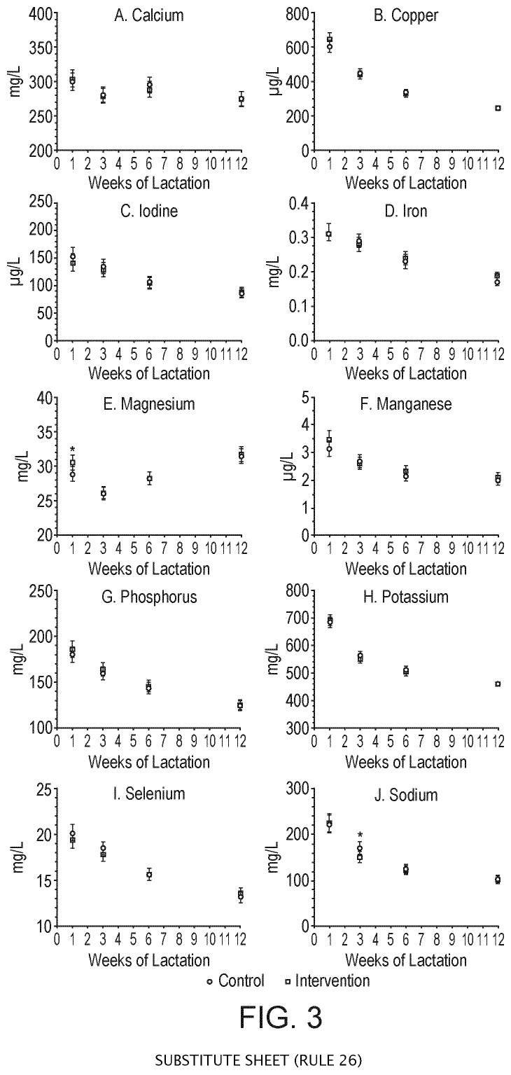

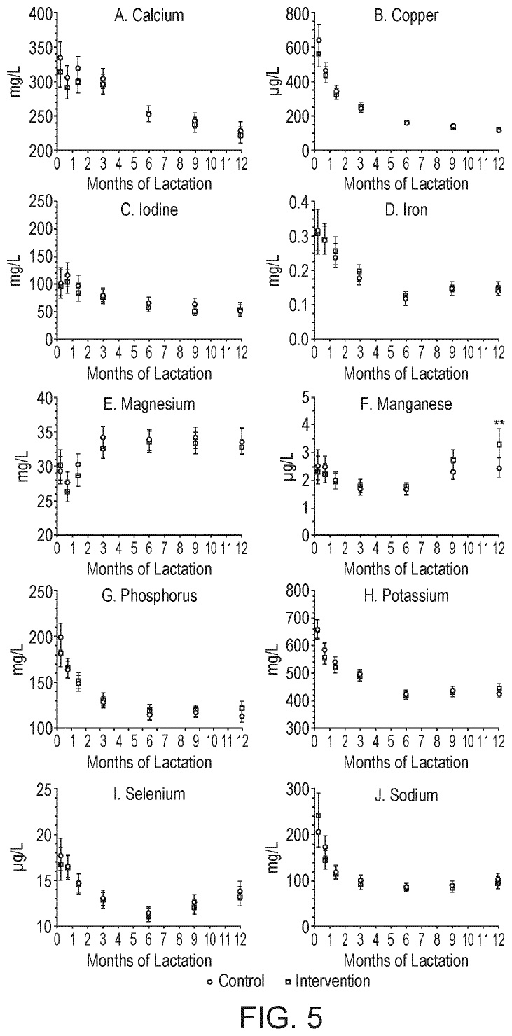

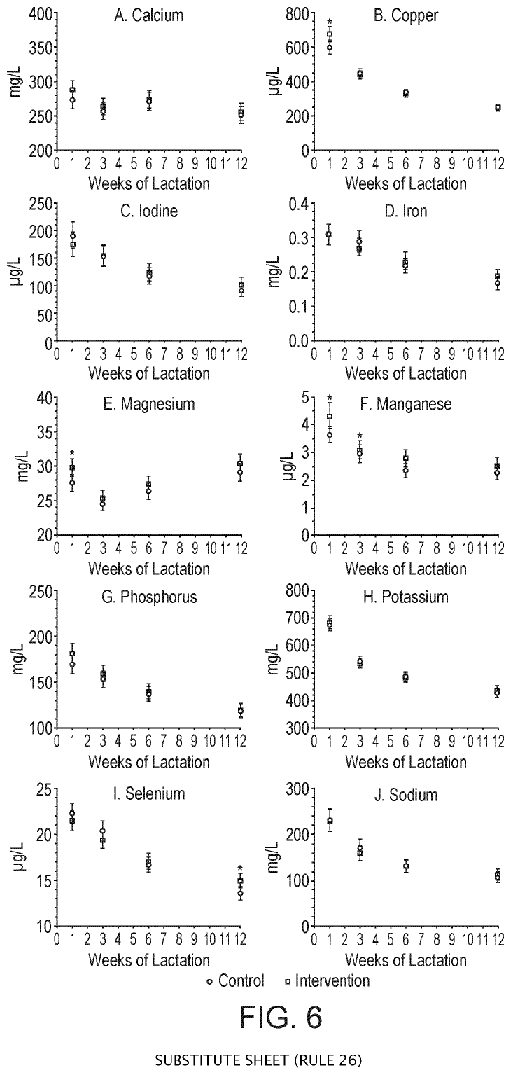

There were no observed intervention effects on other HM minerals analysed in the first 3 months of lactation (FIGS. 3A-3J). The exceptions were isolated (and likely random) findings on magnesium at 1 week (FIG. 3E) and sodium at 3 weeks (FIG. 3J); in New Zealand, on manganese at 12 months (FIG. 5F); in Singapore on copper at 1 week (FIG. 6B), magnesium at 1 week (FIG. 6E), manganese on 1 and 3 weeks (FIG. 6F) and, selenium at 12 weeks (FIG. 6I).

### Changes in Minerals Over Time in New Zealand (0-12 Months)

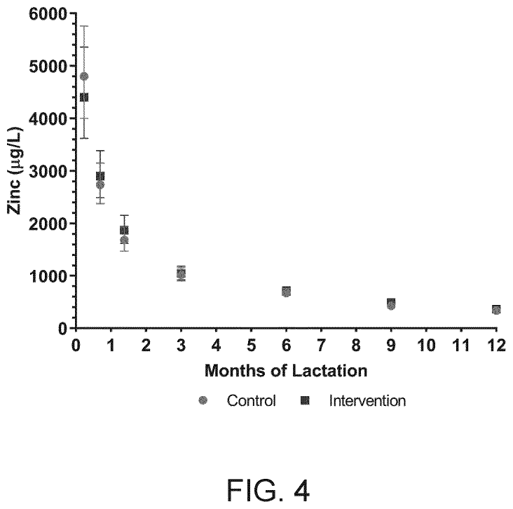

Zinc concentrations in HM in New Zealand decreased markedly over the first 3 months of lactation and continued to decline until 12 months (FIG. 4; Table 12). In both control and intervention groups, zinc concentration peaked at 1 week [4801 (5759, 4002) and 4405 (5358, 3621) μg/L, respectively], with approximately 4.5-fold reduction by 3 months [1021 (1157, 901) and 1048 (1191, 923) μg/L, respectively](FIG. 4). As zinc concentrations continued to decline, their nadir was reached at 12 months at the end of our HM collection period [340 (392, 295) and 365 (423, 315) μg/L, respectively](FIG. 4).

TABLE 12
Overall mineral concentrations in human milk of participants in New
Zealand in the NiPPeR study, during the first 12 months of lactation.

| Mineral | 1 week | 3 weeks | 6 weeks | 3 months | 6 months | 9 months | 12 months |
|---|---|---|---|---|---|---|---|
| Zinc | 4599 (4023, | 2820 (2541, | 1778 (1611, | 1035 (946, | 686 (632, | 458 (421, | 352 (318, |
| (μg/L) | 5257) | 3129) | 1963) | 1131) | 745) | 498) | 390) |
| Calcium | 324 (308, | 298 (287, | 309 (298, | 300 (290, | 253 (245, | 240 (232, | 225 (216, |
| (mg/L) | 341) | 310) | 321) | 311) | 261) | 248) | 234) |
| Copper | 600 (544, | 450 (418, | 335 (313, | 248 (232, | 160 (151, | 138 (130, | 116 (108, |
| (μg/L) | 662) | 484) | 360) | 264) | 169) | 146) | 125) |
| Iodine | 100 (84, | 110 (97, | 91 (80, | 79 (70, | 62 (56, | 58 (52, | 54 (47, |
| (μg/L) | 120) | 126) | 104) | 89) | 69) | 64 | 62) |
| Iron | 0.31 (0.27, | 0.29 (0.26, | 0.25 (0.23, | 0.19 (0.17, | 0.12 (0.11, | 0.15 (0.14, | 0.15 (0.13, |
| (mg/L) | 0.36) | 0.32) | 0.28) | 0.20) | 0.13) | 0.16) | 0.16) |
| Magnesium | 29.7 (28.3, | 27.1 (26.0, | 29.4 (28.3, | 33.3 (32.3, | 33.6 (32.6, | 33.7 (32.6, | 33.5 (32.3, |
| (mg/L) | 31.2) | 28.1) | 30.5) | 34.5) | 34.7) | 34.8) | 34.8) |
| Manganese | 2.40 (2.07, | 2.34 (2.10, | 1.96 (1.77, | 1.73 (1.58, | 1.68 (1.54, | 2.50 (2.29, | 2.82 (2.52, |
| (μg/L) | 2.79) | 2.61) | 2.18) | 1.90) | 1.83) | 2.73) | 3.14) |
| Phosphorus | 190 (180, | 165 (158, | 150 (144, | 130 (125, | 118 (114, | 119 (115, | 118 (113, |
| (mg/L) | 201) | 172) | 157) | 135) | 122) | 123) | 123) |
| Potassium | 658 (634, | 570 (554, | 530 (516, | 493 (481, | 423 (414, | 433 (423, | 435 (423, |
| (mg/L) | 683) | 587) | 545) | 505) | 433) | 443) | 447) |
| Selenium | 17.2 (16.0, | 16.4 (15.6, | 14.6 (13.9, | 12.9 (12.3, | 11.3 (10.8, | 12.3 (11.8, | 13.5 (12.8, |
| (μg/L) | 18.5) | 17.3) | 15.4) | 13.5) | 11.8) | 12.8) | 14.2) |
| Sodium | 224 (197, | 158 (144, | 116 (106, | 95 (88, | 85 (79, | 86 (80, | 98 (89, |
| (mg/L) | 254) | 174) | 128) | 103) | 92) | 93) | 108) |

Data are the least-squares means (i.e. adjusted means) at each time point, adjusted for randomisation group, visit, their interaction term (group*visit), adherence, and maternal pre-pregnancy body mass index.

For other minerals, different patterns of change were observed over time (Table 12). Calcium and copper concentrations progressively decreased throughout the first 12 months of lactation, while iodine remained stable throughout the study period (Table 12). Concentrations of iron, potassium, sodium, and phosphate gradually declined until 6 months, but then remained relatively constant until 12 months (Table 12). In contrast, magnesium concentrations increased during the first 3 months of lactation but were largely unchanged thereafter (Table 12). The concentrations of manganese and selenium followed a U-shape pattern with a nadir observed at 6 months. (Table 12).

### Impact of Preconception and Pregnancy Intervention on Human Milk Vitamin D

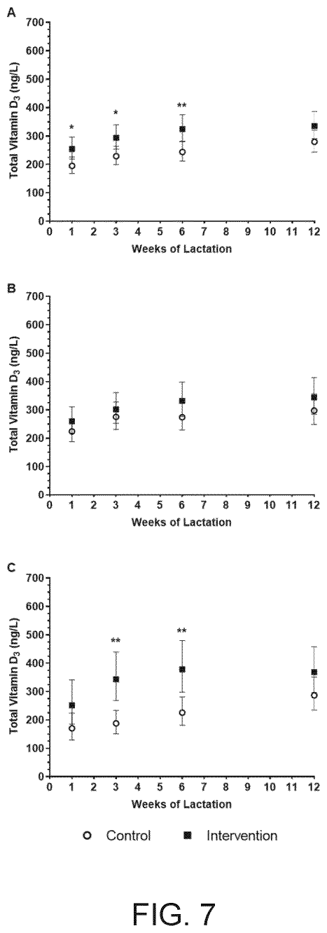

Over the first 3 months of lactation, total HM vitamin D₃ concentrations were 28% higher in the intervention group compared to the control group (p=0.003, Table 13). When total vitamin D₃ concentrations at individual visits were examined, the difference between the groups was more evident at 1 week, 3 weeks, and 6 weeks, the intervention group being higher by 31% (p=0.013), 28% (p=0.014) and 33% (p=0.006), respectively, than the control group (FIG. 7A). Such intervention effect was also reflected in both vitamin D₃ and 25(OH)D₃ concentrations. The vitamin D₃ concentrations was higher by 46% (p=0.007) and 25(OH)D₃ concentrations by 17% (p=0.002) in the intervention group over the first 3 months of lactation (Table 13).

TABLE 13
Comparisons of vitamin D concentrations in human milk (HM) over the
first 3 months of lactation in intervention and control groups.

| Vitamin D (ng/L) | Intervention | Control | aMD | p-value |
|---|---|---|---|---|
| Total Vitamin D₃ | 300 (267, | 235 (210, | 1.28 (1.09, | 0.003 |
| | 337) | 263) | 1.50) | |
| Singapore | 302 (263, | 266 (231, | 1.13 (0.93, | 0.217 |
| | 348) | 307) | 1.39) | |
| New Zealand | 331 (275, | 213 (180, | 1.55 (1.21, | 0.001 |
| | 399) | 253) | 1.99) | |
| Vitamin D₃ | 125 (102, | 86 (71, | 1.46 (1.11, | 0.007 |
| | 152) | 104) | 1.91) | |
| Singapore | 131 (103, | 108 (84, | 1.22 (0.86, | 0.271 |
| | 168) | 138) | 1.74) | |
| New Zealand | 134 (98, | 67 (50, | 2.01 (1.32, | 0.001 |
| | 184) | 88) | 3.07) | |
| 25(OH)D₃ | 135 (125, | 115 (107, | 1.17 (1.06, | 0.002 |
| | 145) | 124) | 1.29) | |
| Singapore | 131 (121, | 121 (112, | 1.08 (0.96, | 0.175 |
| | 142) | 131) | 1.21) | |
| New Zealand | 153 (135, | 117 (104, | 1.31 (1.11, | 0.001 |
| | 173) | 130) | 1.55) | |

Data are the least-square mean (i.e., adjusted mean) for each group or the adjusted mean difference (aMD) and respective 95% confidence intervals derived from repeated measures analyses, adjusted for visit, an interaction term (group*visit), study site, adherence, maternal pre-pregnancy body mass index, baseline serum vitamin D₃ concentrations and season. All data have been log-transformed to approximate a normal distribution, and then, back-transformed, so the aMD represents a proportional difference between the groups (i.e., intervention vs control). Bold font indicates a statically significant difference between groups (at p < 0.05).

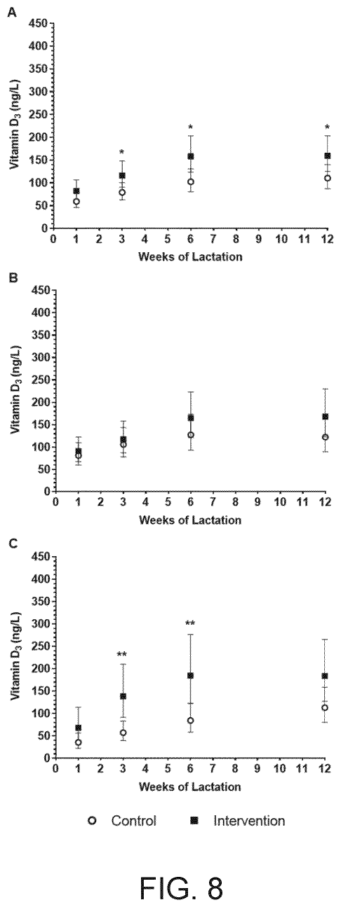

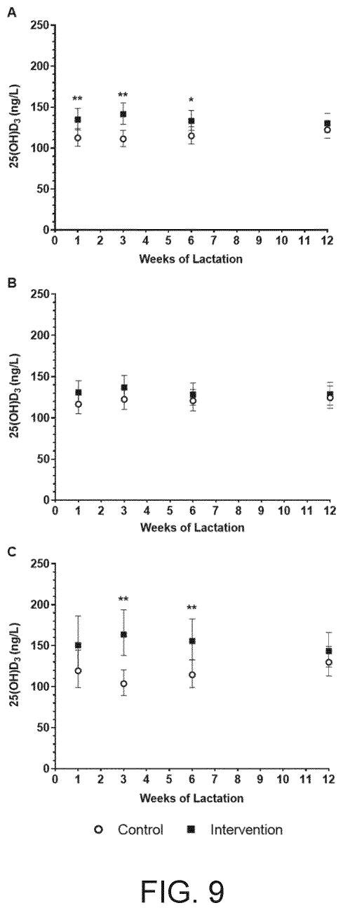

In analyses stratified by site, in New Zealand, total HM vitamin D₃ concentrations was higher in the intervention group by 55% over the first 3 months of lactation compared to the control group (Table 13). The difference between the groups were more evident at 3 weeks [intervention 343 (95% CI 268, 439) ng/L vs control 188 (95% CI 151, 234) ng/L, p<0.001] and at 6 weeks [intervention 378 (95% CI 298, 480) vs control 226 (95% CI 181, 281) ng/L, p=0.002](FIG. 7C). A similar pattern was observed in for vitamin D₃ at 3 weeks (FIG. 8C) and for 25(OH)D₃ at 6 weeks (FIG. 9C) in New Zealand (Table 13). In Singapore, no differences in HM vitamin D₃ (FIG. 8B) and 25(OH)D₃ (FIG. 9B) concentrations were detected, although the overall pattern of the groups was also observed at that site.

### Impact of Preconception and Pregnancy Intervention on Human Milk Vitamin B

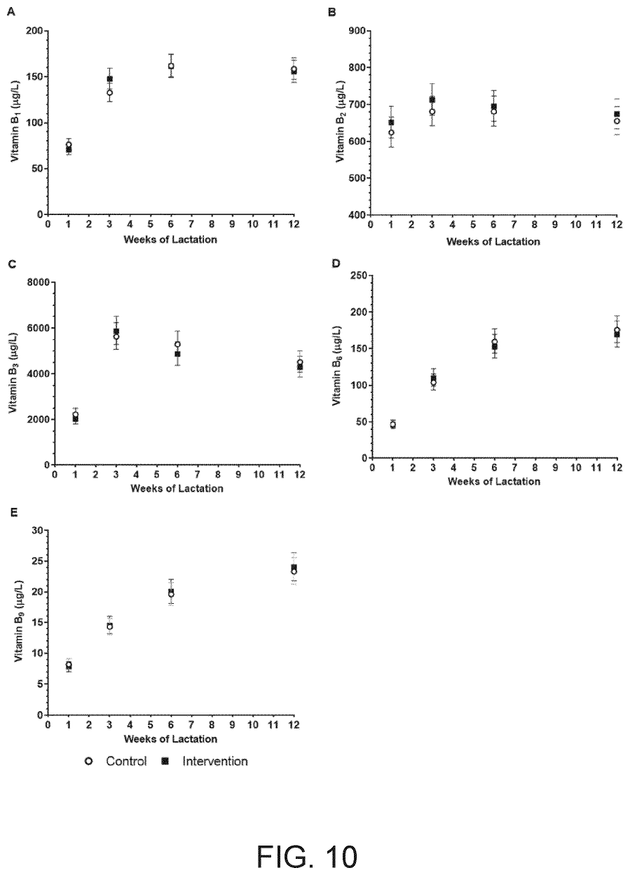

Overall, the total HM vitamin B1, B₂, B₃, B₆ and B₉ concentrations was not different between the control and the intervention groups during the first 3 months of lactation (Table 14) or at any individual time points (FIG. 10). Among the B-vitamers examined, TMP was higher in the intervention group compared to the control group at 3 weeks of lactation (p=0.021, Table 15).

TABLE 14
Comparisons of vitamin B concentrations in human milk (HM) over the
first 3 months of lactation in intervention and control groups.

| Vitamin | | | | |
|---|---|---|---|---|
| B (μg/L) | Intervention | Control | aMD | p-value |
| B₁ | Significant group*visit interaction p = 0.012 | | | |
| B₂ * | 683 | 660 | 1.035 | 0.228 |
| | (656, 711) | (634, 686) | (0.979, 1.094) | |
| B₃ | 3975 | 4162 | 0.955 | 0.384 |
| | (3686, 4286) | (3867, 4480) | (0.861, 1.060) | |
| B₆ * | 107 | 108 | 0.991 | 0.865 |
| | (99, 116) | (100, 117) | (0.891, 1.102) | |
| B₉ ^{†} | 15.3 | 15.2 | 1.002 | 0.972 |
| | (14.2, 16.4) | (14.2, 16.3) | (0.909, 1.104) | |

* Vitamin B present only in the intervention drink.
^{†} Vitamin B present in both control and intervention drinks. Data are the least-square mean (i.e., adjusted mean) for each group or the adjusted mean difference (aMD) and respective 95% confidence intervals derived from repeated measures analyses, adjusted for visit, an interaction term (group*visit), study site, adherence, infant gestational age and maternal pre-pregnancy body mass index. All data have been log-transformed to approximate a normal distribution, and then back-transformed, so the aMD represents a proportional difference between the groups (i.e., intervention vs control).

TABLE 15
Comparisons in average B-vitamer concentrations in human milk (HM) over
the first 3 months of lactation in intervention and control groups.

| Vitamin | B-vitamer | | | | |
|---|---|---|---|---|---|
| B group | (μg/L) | Intervention | Control | aMD | p-value |
| B₁ | Thiamine | 16.0 (14.5, 17.5) | 15.3 (14.0, 16.8) | 1.042 (0.914, 1.188) | 0.538 |
| | TMP | Significant Group*Visit interaction p = 0.008 | | | |
| | 1 week | 65 (57, 71) | 58(52, 65) | 0.918 (0.785, 1.074) | 0.287 |
| | 3 weeks | 106 (96, 118) | 126 (113, 140) | 1.186 (1.026, 1.371) | 0.021 |
| | 6 weeks | 125 (112, 138) | 129 (116, 143) | 1.033 (0.892, 1.195) | 0.666 |
| | 3 months | 110 (99, 122) | 109 (98, 121) | 0.995 (0.861, 1.150) | 0.942 |
| | TPP | 3.89 (3.58, 4.21) | 3.94 (3.64, 4.27) | 0.986 (0.881, 1.103) | 0.802 |
| B₂ * | FAD | 574 (551, 599) | 548 (526, 571) | 1.048 (0.990, 1.110) | 0.109 |
| | FMN | 21.9 (19.8, 24.2) | 21.0 (19.0, 23.1) | 1.045 (0.911, 1.198) | 0.530 |
| | Riboflavin | 52.1 (45.3, 59.8) | 46.2 (40.3, 53.0) | 1.126 (0.928, 1.366) | 0.228 |
| B₃ | Nicotinamide | 229 (208, 251) | 222 (203, 244) | 1.027 (0.903, 1.169) | 0.679 |
| | NMN | 3223 (2933, 3538) | 3378 (3083, 3701) | 0.954 (0.839, 1.085) | 0.473 |
| | NRT | 189 (155, 230) | 208 (172, 253) | 0.906 (0.691, 1.189) | 0.477 |
| B₆ * | Pyridoxal | 76.0 (69.2, 83.5) | 74.4 (67.9, 81.6) | 1.022 (0.897, 1.163) | 0.746 |
| | PLP | 23.6 (21.6, 25.8) | 24.7 (22.6, 27.0) | 0.955 (0.844, 1.081) | 0.465 |
| | PMP | 1.48 (1.35, 1.61) | 1.39 (1.28, 1.52) | 1.059 (0.936, 1.199) | 0.364 |
| B₉ ^{†} | 5MeTHF | 9.52 (8.92, 10.15) | 8.91 (8.37, 9.49) | 1.068 (0.977, 1.168) | 0.145 |
| Individual | NAD | 1760 (1655, 1873) | 1780 (1676, 1892) | 0.989 (0.908, 1.077) | 0.792 |
| vitamers | NADP | 168 (155, 183) | 160 (148, 174) | 1.050 (0.936, 1.178) | 0.406 |

In Singapore, there were no differences in IM B-vitamins and B-vitamers (data not shown) concentrations between the control and intervention groups over the first 3 months.

In New Zealand, several differences in IM vitamin B₆ and some B-vitamer concentrations between the control and intervention groups were detected in early lactation.

Vitamin B₆ concentrations was higher in the control group at 1 week [intervention 31.0 (95% CI 25.2, 38.1) μg/L vs control 46.4 (95% CI 38.6, 55.7) μg/L, p=0.005] which was also reflected by higher PLP concentration in the control group at the same time point [intervention 14.0 (95% CI 10.5, 18.6) μg/L vs control 23.1 (95% CI 17.8, 30.1) μg/L, p=0.011].

Thiamine [intervention 15.09 (95% CI 12.34, 18.45) μg/L vs control 11.05 (95% CI 9.20, 13.26) μg/L, p=0.025] and TMP [intervention 132 (95% CI 114, 154) μg/L vs control 106 (95% CI 92, 121) μg/L, p=0.031], both vitamers for vitamin B₁, were higher in the intervention group at 3 weeks.

Although NM and NMN are both vitamers for vitamin B₃, NM was higher in the intervention group at 1 week [intervention 247 (95% CI 190, 321) μg/L vs control 168 (95% CI 132, 214) μg/L, p=0.034] but NMN was higher in the control group at 1 week [intervention 1559 (95% CI 1186, 2048) μg/L vs control 2306 (95% CI 1790, 2970) μg/L, p=0.040]. NAD was also higher in the intervention group at 1 week [intervention 1262 (95% CI 1018, 1563) μg/L vs control 2174 (95% CI 1783, 2652) μg/L, p<0.001].

Finally, 5MeTHF concentration was higher in the intervention group at 3 weeks of lactation [intervention 9.4 (95% CI 8.1, 10.9) μg/L vs control 6.9 (95% CI 6.1, 7.9) μg/L, p=0.003].

### Changes in Human Milk Vitamins Over Time in New Zealand (0-12 Months)

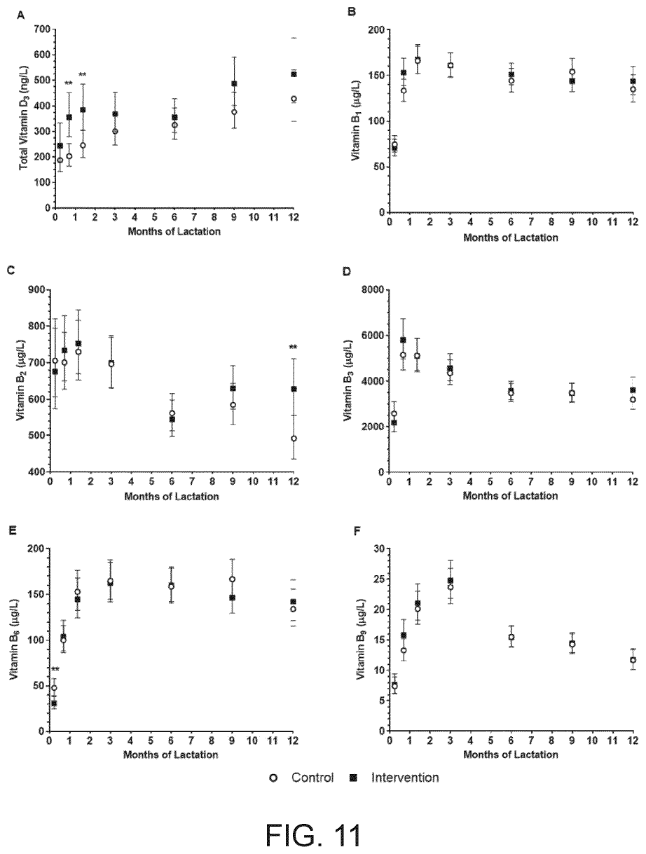

In New Zealand, HM vitamin concentrations changed dynamically over the first 12 months of lactation. Total HM vitamin D₃ concentrations gradually increased from 1 week to 12 months of lactation (Table 16), and the pattern was similar in both the control and intervention groups (FIG. 11A). In the control group, total vitamin D₃ concentrations increased from 188 (95% CI 143, 248) ng/L at 1 week to 429 (95% CI 340, 541) ng/L at 12 months. In the intervention group, total vitamin D₃ concentrations increased from 244 (95% CI 179, 333) ng/L to 524 (95% CI 412, 666) ng/L from 1 week to 12 months of lactation. This increase in total vitamin D₃ concentrations was reflected in HM vitamin D₃ and 25(OH)D₃, both of which gradually increased over the same time period (Table 16).

TABLE 16
Overall vitamin D concentrations in human milk of participants in New
Zealand in the NiPPeR study, during the first 12 months of lactation.

| Vitamin D | | | | | | | |
|---|---|---|---|---|---|---|---|
| (ng/L) | 1 week | 3 weeks | 6 weeks | 3 months | 6 months | 9 months | 12 months |
| Total Vitamin | 214 (174, | 269 (229, | 308 (263, | 333 (288, | 340 (298, | 428 (375, | 474 (401, |
| D₃ | 264) | 317) | 360) | 385) | 388) | 490) | 560) |
| Vitamin D₃ | 52 (37, | 98 (76, | 132 (103, | 149 (118, | 158 (128, | 209 (169, | 241 (184, |
| | 72) | 128) | 170) | 187) | 195) | 259) | 315) |
| 25(OH)D₃ | 137 (117, | 132 (117, | 136 (121, | 139 (125, | 147 (134, | 173 (157, | 179 (158, |
| | 159) | 148) | 152) | 154) | 162) | 190) | 202) |

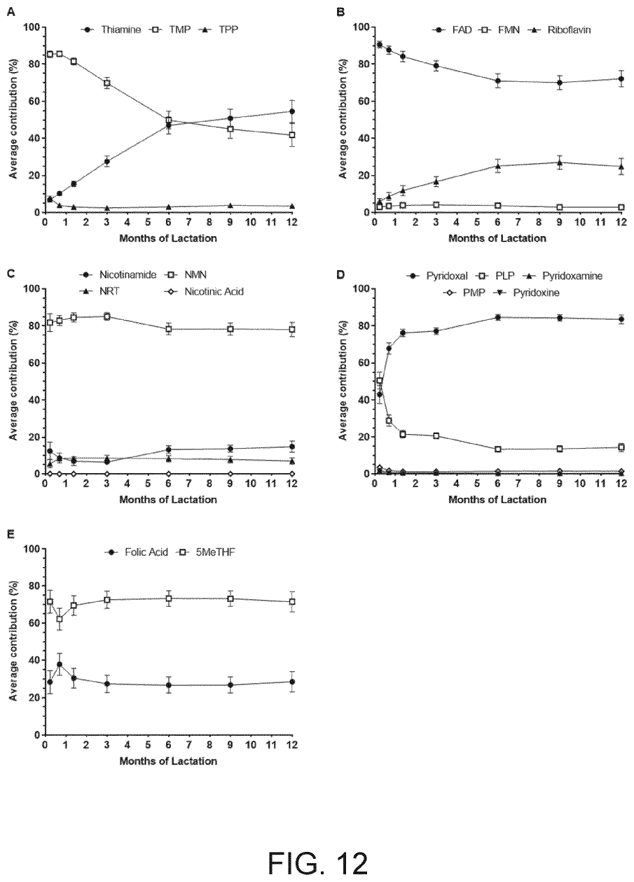

Total vitamin B₁ concentrations in New Zealand increased from 1 week to 6 weeks of lactation then remained constant thereafter until 12 months (Table 17). TMP concentrations followed a similar pattern, peaking at 6 weeks then decreasing until 12 months (Table 17). During this time, TMP contribution to total vitamin B₁ decreased from 85.4% at 1 week to 41.8% at 12 months (FIG. 12A). In contrast, thiamine concentrations continued to gradually increase from 1 week to 12 months of lactation (Table 17), increasing its contribution to total vitamin B₁ from 7.0% at 1 week to 54.6% at 12 months (FIG. 12A).

TABLE 17
Overall vitamin B concentrations in human milk of participants in New
Zealand in the NiPPeR study, during the first 12 months of lactation.

| Vitamin B | | | | | | | |
|---|---|---|---|---|---|---|---|
| (μg/L) | 1 week | 3 weeks | 6 weeks | 3 months | 6 months | 9 months | 12 months |
| Vitamin B₁ | 73 (66, | 143 (134, | 167 (156, | 161 (152, | 148 (139, | 149 (140, | 139 (129, |
| | 79) | 153) | 178) | 171) | 157) | 158) | 150) |
| Thiamine | 4.4 (3.8, | 12.9 (11.4, | 23.9 (21.3, | 40.0 (36.1, | 59.0 (53.7, | 67.5 (61.2, | 71.1 (63.2, |
| | 5.2) | 14.5) | 26.8) | 44.4) | 64.9) | 74.4) | 80.0) |
| TMP | 62 (51. | 119 (102, | 132 (115, | 108 (95, | 64 (57, | 52 (46, | 44 (38, |
| | 76) | 138) | 152) | 123) | 72) | 59) | 51) |
| TPP | 4.60 (3.88, | 4.59 (4.04, | 4.11 (3.63, | 3.48 (3.11, | 3.48 (3.11, | 4.66 (4.14, | 4.41 (3.81, |
| | 5.46) | 5.22) | 4.66) | 3.89) | 3.90) | 5.24) | 5.10) |
| Vitamin B₂ * | 691 (618, | 718 (661, | 741 (684, | 698 (650, | 554 (519, | 607 (568, | 556 (510, |
| | 772) | 779) | 804) | 750) | 591) | 649) | 607) |
| FAD | 621 (550, | 623 (570, | 615 (563, | 539 (499, | 368 (343, | 408 (380, | 395 (361, |
| | 700) | 681) | 670) | 582) | 394) | 438) | 432) |
| FMN | 16.2 (12.8, | 21.8 (18.1, | 22.9 (19.2, | 21.7 (18.5, | 13.6 (11.7, | 10.2 (8.8, | 8.5 (7.1, |
| | 20.6) | 26.3) | 27.3) | 25.5) | 15.7) | 11.8) | 10.3) |
| Riboflavin | 36 (27, | 43 (34, | 55 (44, | 85 (70, | 102 (85, | 121 (102, | 99 (79, |
| | 47) | 53) | 67) | 102) | 121) | 145) | 123) |
| Vitamin B₃ | 2377 (2076, | 5467 (4932, | 5115 (4633, | 4470 (4088, | 3531 (3258, | 3485 (3207, | 3401 (3070, |
| | 2723) | 6061) | 5646) | 4888) | 3826) | 3788) | 3769) |
| Nicotinamide | 200 (169, | 335 (295, | 264 (233, | 232 (207, | 360 (326, | 389 (351, | 425 (374, |
| | 237) | 381) | 298) | 259) | 398) | 431) | 483) |
| NMN | 1908 (1575, | 4450 (3851, | 4189 (3645, | 3781 (3338, | 2656 (2374, | 2613 (2328, | 2534 (2193, |
| | 2312) | 5143) | 4814) | 4283) | 2971) | 2934) | 2928) |
| NRT | 55 (36, | 330 (239, | 278 (203, | 213 (161, | 129 (101, | 109 (84, | 122 (88, |
| | 85) | 457) | 380) | 282) | 167) | 142) | 170) |
| Vitamin B₆ * | 38 (33, | 102 (91, | 149 (134, | 164 (149, | 159 (147, | 156 (143, | 138 (124, |
| | 44) | 114) | 165) | 180) | 173) | 170) | 154) |
| Pyridoxal | 18 (15, | 70 (64, | 115 (102, | 130 (116, | 137 (124, | 134 (121, | 117 (103, |
| | 22) | 80) | 131) | 146) | 152) | 149) | 134) |
| PLP ¹ | 18.0 (14.5, | 26.0 (22.1, | 28.8 (24.6, | 30.8 (26.7, | 16.9 (14.9, | 16.1 (14.2, | 14.9 (12.6, |
| | 22.4) | 30.6) | 33.7) | 35.5) | 19.2) | 18.4) | 17.5) |
| PMP | 1.35 (1.15, | 1.62 (1.43, | 1.63 (1.45, | 1.59 (1.43, | 1.66 (1.50, | 1.69 (1.53, | 1.56 (1.38, |
| | 1.58) | 1.83) | 1.83) | 1.77) | 1.83) | 1.87) | 1.76) |
| Vitamin B₉ ^{†} | 7.5 (6.6, | 14.5 (13.1, | 20.6 (18.7, | 24.2 (22.2, | 15.5 (14.3, | 14.4 (13.3, | 11.7 (10.6, |
| | 8.7) | 16.1) | 22.7) | 26.5) | 16.8) | 15.6) | 13.0) |
| 5MeTHF | 5.04 (4.33, | 8.03 (7.19, | 13.3 (11.9, | 16.3 (14.8, | 10.6 (9.76, | 9.86 (9.02, | 7.95 (7.11, |
| | 5.86) | 8.97) | 14.7) | 17.9) | 11.6) | 10.8) | 8.88) |

Individual vitamers

| | | | | | | | |
|---|---|---|---|---|---|---|---|
| NAD | 1663 (1420, | 2129 (1891, | 1825 (1629, | 1816 (1641, | 1743 (1593, | 2307 (2100, | 2291 (2033, |
| | 1947) | 2397) | 2046) | 2010) | 1908) | 2535) | 2581) |
| NADP | 133 (106, | 195 (165, | 223 (189, | 233 (201, | 150 (132, | 199 (174, | 170 (143, |
| | 167) | 231) | 262) | 269) | 171) | 228) | 201) |

* Vitamin B present only in the intervention drink.
^{†} Vitamin B present in both control and intervention drinks.
¹ A significant group*visit interaction term, p = 0.035. Data are the least square means (i.e., adjusted means) and respective 95% confidence intervals at each time point, adjusted for randomisation group, visit, their interaction term (group*visit), adherence, infant gestational age and maternal pre-pregnancy body mass index. All data have been log-transformed to approximate a normal distribution, and then back-transformed. Abbreviations: 5MeTHF, 5-methyl tetrahydrofolic acid; FAD, flavin adenine dinucleotide; FMN, flavin mononucleotide; NAD, nicotinamide adenine dinucleotide; NAPD, nicotinamide adenine dinucleotide phosphate; NMN, nicotinamide mononucleotide; NRT, nicotinamide riboside; PLP, pyridoxal 5′-phosphate; PMP, pyridoxamine-5′-phsophate; TMP, thiamine monophosphate; TPP, thiamine pyrophosphate.

Total vitamin B₂ concentrations increased from 1 week to 6 weeks then after a slight decrease by 6 months, showed an increasing trend from 6 months to 12 months of lactation in New Zealand (Table 17). This pattern was also displayed by FAD, peaking at 3 weeks of lactation. On the other hand, riboflavin concentrations gradually increased from 1 week to 9 months of lactation (Table 17). As such, there was a change in contribution of these vitamers to total vitamin B₂ during this time. As FAD contribution decreased from 90.6% to 70.1%, riboflavin contribution increased from 6.2% to 27.0% from 1 week to 9 months of lactation (FIG. 12B).

Total vitamin B₃ concentration reached the highest at 3 weeks and the lowest at 6 months then remained stable from 6 months of 12 months of lactation (Table 9). This pattern was also reflected in NMN concentrations which peaked at 3 weeks then gradually decreased until 12 months of lactation (Table 17). NMN was the predominant vitamer for vitamin B₃ over the first 12 months of lactation, its contribution ranging from 78.1% to 85.1% (FIG. 12C).

Total vitamin B₆ concentration increased from 1 week to 3 months then remained stable thereafter until 12 months of lactation (Table 17). Pyridoxal concentrations showed a similar pattern, reaching a maximum at 6 months of lactation (Table 17). PLP concentrations showed an increasing trend from 1 week to 3 months of lactation then steadily decreased from 3 months to 12 months of lactation (Table 17). In early lactation, PLP was the predominant vitamer for vitamin B₆, contributing 50.6%, while pyridoxal contributed 43.0%. By 12 months of lactation, this ratio shifted, PLP contribution decreasing to 14.5% and pyridoxal contribution increasing to 83.6% (FIG. 12D).

Finally, total vitamin B₉ concentrations increased in early lactation, reaching the maximum at 3 months. Then following a steep decrease from 3 months to 6 months, its concentrations remained constant from 6 months to 12 months of lactation (Table 17). This pattern was also reflected in 5MeTHF, reaching the highest concentration at 3 months (Table 17). Throughout the first 12 months of lactation, 5MeTHF remained to be predominant contributor to vitamin B₉, ranging from 62.2% to 73.3%, and folic acid contribution ranged from 26.7% to 37.8% (FIG. 12E).

### Discussion—Zinc

The present study showed that the NiPPeR intervention supplement containing zinc taken before and during pregnancy increased HM zinc concentrations. Overall, the effect was more evident at 6 weeks and persisted throughout the first 3 months of lactation. This effect was more evident in Singapore than in New Zealand, possibly due to a smaller sample size early in lactation at the latter site.

Previous studies have not examined the impact of zinc supplementation prior to as well as during pregnancy on HM zinc concentrations. The majority of intervention studies begin well into the first trimester of pregnancy. A study in Indonesian women from rural areas observed no effects of higher-dose zinc supplementation (30 mg/day) during pregnancy (recruited before 20 weeks of gestational age), either alone or combined with β-carotene, on HM zinc concentrations, measured in the first and sixth months of lactation (Am J Clin Nutr. 2004; 80(5):1299-307). In contrast, in the current study, we demonstrated a 19% increase in HM zinc concentrations at 6 weeks of lactation as a result of zinc supplementation (10 mg/day) before and during pregnancy. Such effects of supplementation may not have been detected in the previous study due to a smaller sample size and variations in HM collection time point. The authors acknowledged that a single HM sample collected at any time across the first month of lactation could have masked the intervention effect (Am J Clin Nutr. 2004; 80(5):1299-307), as zinc concentrations dynamically change during this time as we have observed (FIG. 2). Nevertheless, both studies indicate that pregnancy zinc supplementation does not influence HM concentrations at 6 months of lactation, as the pregnancy intervention effect would be expected to decline postnatally over time.

HM zinc concentrations progressively decline throughout lactation. Previously, Silvestre et al. showed that zinc concentration was the highest in colostrum at 7990±3230 g/L which decreased to 1050±710 g/L by day 90 (Biol Trace Elem Res. 2001; 80(1):1-11). Similarly, Djurovic et al. reported a decrease in zinc concentration from 4700±1740 μg/L at day 1 to 460±360 μg/L at 6 months (Analyst. 1995; 120(3):895-7). These concentrations are comparable to those in our study at the respective time points. In our study, beyond 6 months, zinc concentrations continued to steadily decrease until 12 months of lactation, which has also been described previously (Am J Clin Nutr. 1989; 49(5):773-85).

Other HM minerals not exclusively in the intervention that were measured through 12 months of lactation were: calcium, copper, iron, magnesium, manganese, phosphorus, potassium, selenium and sodium. Across these HM minerals, quite contrasting patterns of changes in concentration over lactation were observed, and may reflect different critical roles at different stages of infancy. Iodine remained relatively constant throughout 12 months, copper progressively decreased while others (iron, potassium, sodium and phosphorus) decreased until 6 months then plateaued. Calcium increased in early lactation then decreased and magnesium increased in early lactation and stabilised. Manganese and selenium had a U-shape pattern during the first 12 months.

Similar to our findings, Sabatier et al. (Nutrients. 2019; 11(8)) observed stable iodine concentrations in the first 8 weeks. However, others have found a decrease in iodine concentrations in healthy Chinese women (Chin Med J (Engl). 2014; 127(14):2643-2648) and in unsupplemented, iodine deficient women (Am J Clin Nutr. 2010; 92(4):849-56). The differences between the studies might be due to the differences in regional dietary iodine intake (Eur J Clin Nutr. 2021; (August)), study population and intervention method. More studies are required to better understand the change in HM iodine concentrations over time.

HM copper concentrations have been found to decrease over time, at least during the first 5 months of lactation (Am J Clin Nutr. 1989; 49(5):773-85; Biol Trace Elem Res. 2001; 79(3):221-33; J Trop Pediatr. 1989; 35(3):126-8). Previous studies have reported that copper concentrations from 6 months to 12 months (J Trop Pediatr. 1989; 35(3):126-8) and 13 months (Am J Clin Nutr. 1989; 49(5):773-85) do not change. Conversely, in our study, with larger sample size, we have observed a small but steady decrease in copper concentrations over the same time period.

HM iron concentrations have been reported to be highest in colostrum followed by a steady decrease through the first 6 months of lactation (Eur J Clin Nutr. 2006; 60(7):903-8; Pediatr Res. 1982; 16(2):113-7). In the current study, we were also able to describe such decreases in HM iron concentrations until 6 months of lactation. Further, beyond this time point, we observed that HM iron concentrations remained stable until 12 months.

A clear decrease in HM potassium and sodium concentrations over time have been demonstrated previously (Pediatr Res. 1982; 16(2):113-7; Nutrition. 1997; 13(9):774-7; J Trop Pediatr. 2006; 52(4):272-5). More specifically, Wack et al. showed that after day 120 of lactation, potassium and sodium concentrations remain stable (Nutrition. 1997; 13(9):774-7). Similar patterns were described in our study where both sodium and potassium concentrations decreased in the first 6 months then remained constant until 12 months of lactation. It These stable concentrations of HM potassium and sodium may play a role in maintaining the osmolarity of the milk, which may influence the nutrient content of the milk and infant electrolyte balances (Nutrition. 1997; 13(9):774-7).

Previous studies on HM phosphorus concentrations have described different patterns of change over time (J Trop Pediatr. 1989; 35(3):126-8; Pediatr Res. 1982; 16(2):113-7; J Pediatr. 1982; 100(1):59-64). While Greer et al. (J Pediatr. 1982; 100(1):59-64) reported a decrease in phosphorus concentration from week 3 to 26 weeks, Lemons et al. (Pediatr Res. 1982; 16(2):113-7) (from day 7 to day 28) and Nagra et al. (J Trop Pediatr. 1989; 35(3):126-8) (from 1 month to 12 months) reported that the concentrations remained constant over time. In our study, similar to Greer et al., we have observed a decreasing pattern in HM phosphorus concentrations throughout the first 6 months, after which it remained stable until 12 months of lactation.

In the present study, an initial increase in HM calcium from 3 weeks to 6 weeks was observed which then steadily decreased until 12 months of lactation. Such rapid increase in HM calcium in early lactation have also been observed previously (J Pediatr. 1982; 100(1):59-64; J Dairy Res. 1992; 59(2):161-7). Similarly, HM magnesium concentrations increased from 3 weeks to 3 months then plateaued. However, others have reported no changes in HM magnesium from 3 weeks to 26 weeks (J Pediatr. 1982; 100(1):59-64) and from 2 weeks to 2 months (J Trop Pediatr. 2006; 52(4):272-5) of lactation. The higher HM concentrations of phosphorus, calcium and magnesium in early lactation may be related to bone formation in infancy (J Pediatr. 1982; 100(1):59-64).

There are limited studies that have investigated changes in HM manganese and selenium concentrations over time. Similar to our study, Casey et al. have observed a decrease in HM manganese from 1 month to 4 months then an increasing trend from 6 months to 13 months (Am J Clin Nutr. 1989; 49(5):773-85). HM selenium concentrations were reported to decrease during the first month of lactation (Biol Trace Elem Res. 2001; 79(3):221-33). We demonstrated that beyond this time, HM selenium continued to decrease until 6 months then increased in later stages of lactation from 6 months to 12 months. How these U-shape patterns of change in HM manganese and selenium are related to the developmental stage of the infant and their implications of infant nutrition requires further investigation.

### Strengths and Limitations

This study investigated the impact of nutritional supplementation during preconception and pregnancy on HM mineral composition during lactation. An international, multicentral design allowed the investigation of HM mineral composition in a large population of diverse ethnic groups. The Singapore and New Zealand study sites have used standardized sample collection, processing, storage and mineral quantification methods, minimizing any potential variations that might have occurred during these processes. In addition, as we have tightly controlled the visit windows, each HM collection time point was at a distinctive stage of lactation making it possible to describe the changes in HM mineral concentration between the different stages of lactation. However, due to logistical constraints, longitudinal samples could not be collected from each participant. To address the imbalance in the number of samples at each time point, a repeated measures design was used for statistical analyses. While maternal diet and use of other supplements during lactation was not considered in this study, previous studies have reported that HM zinc concentrations are not associated with dietary zinc intake. Therefore, other sources of zinc are expected to have minimal impact on HM concentrations in the current study.

### Conclusions

We have observed that NiPPeR intervention supplement containing zinc taken during preconception and pregnancy was effective in increasing HM zinc concentrations. Further studies are required to assess the impact of the supplement on HM nutrient composition and how that will influence both short- and long-term infant outcomes. Further, with longitudinal HM samples collected until 12 months in New Zealand, we were able to describe the different behavior of various HM minerals over the course of lactation.

### Discussion—Vitamins

The present study showed that the NiPPeR intervention supplement containing 400 IU vitamin D₃ taken before and during pregnancy increased HM total vitamin D₃ concentrations. Overall, the effect was more evident at 1, 3, and 6 weeks post-partum and persisted throughout the first 3 months of lactation. The effect was more evident in New Zealand than in Singapore, with greater vitamin D₃ and 25(OH)D₃ concentrations at 3 weeks and 6 weeks in the intervention group. There were no effects of the NiPPeR intervention supplement on total HM vitamins B₁, B₂, B₃, B₆, or B₉.

HM vitamin D concentration is highly correlated with maternal vitamin D status (Mohamed H J J, Rowan A, Fong B, Loy S L. Maternal serum and breast milk vitamin D levels: Findings from the Universiti Sains Malaysia pregnancy cohort study. PLoS One. 2014; 9(7):3-10; and Streym S V, Højskov C S, Moller U K, Heickendorff L, Vestergaard P, Mosekilde L, et al. Vitamin D content in human breast milk: A 9-mo follow-up study. Am J Clin Nutr. 2016; 103(1):107-14). The major form of vitamin D found in maternal circulation is 25(OH)D and that in HM is the precursor, vitamin D3 (cholecalciferol) (Hollis B W, Wagner C L. The Vitamin D requirement during human lactation: the facts and IOM's “utter” failure. Public Heal Nutr. 2011; 14(4):748-9; and HOLLIS BW, PITTARD III WB, REINHARDT TA. Relationships among Vitamin D, 25-Hydroxyvitamin D, and Vitamin D-Binding Protein Concentrations in the Plasma and Milk of Human Subjects*. J Clin Endocrinol Metab. 1986 Jan. 1; 62(1):41-4). Adequate vitamin D status is essential during pregnancy (Wagner C L, Taylor S N, Johnson D D, Hollis B W. The role of vitamin D in pregnancy and lactation: Emerging concepts. Women's Heal. 2012; 8(3):323-40; and Mulligan M L, Felton S K, Riek A E, Bernal-Mizrachi C. Implications of vitamin D deficiency in pregnancy and lactation. Am J Obstet Gynecol. 2010; 202(5):429.el-429.e9) and previous research has demonstrated improved maternal and infant vitamin D status with supplementation in mothers during these times (Hollis B W, Wagner C L, Howard C R, Ebeling M, Shary J R, Smith P G, et al. Maternal versus infant Vitamin D supplementation during lactation: A randomized controlled trial. Pediatrics. 2015; 136(4):625-34; and Hollis B W, Johnson D, Hulsey TC, Ebeling M, Wagner C L. Vitamin D supplementation during pregnancy: Double-blind, randomized clinical trial of safety and effectiveness. J Bone Miner Res. 2011; 26(10):2341-57). Further, supplementation with high vitamin D dosage during lactation alone can further lead to increased HM vitamin D concentrations (Wagner C L, Hulsey T C, Fanning D, Ebeling M, Hollis B W. High-dose vitamin D3 supplementation in a cohort of breastfeeding mothers and their infants: a 6-month follow-up pilot study. Breastfeed Med. 2006; 1(2):59-70). However, only a few studies have reported its effects on HM vitamin D concentrations when used during pregnancy (Mohamed H J J, Rowan A, Fong B, Loy S L. Maternal serum and breast milk vitamin D levels: Findings from the Universiti Sains Malaysia pregnancy cohort study. PLoS One. 2014; 9(7):3-10; and Wall C R, Stewart A W, Camargo C A, Scragg R, Mitchell E A, Ekeroma A, et al. Vitamin D activity of breast milk in women randomly assigned to Vitamin D3 supplementation during pregnancy. Am J Clin Nutr. 2016; 103(2):382-8). In a New Zealand double-blind placebo-controlled trial, pregnant women were recruited at 27 weeks of gestation and randomised to receive either the placebo, 1,000 IU, or 2,000 IU vitamin D₃ per day until 36 weeks of gestation. HM samples collected at 2 weeks and 2 months post-partum were analysed for total vitamin D concentration and were higher in the 2,000 IU group, compared to the 1,000 IU group, for both time points. Similarly, an observational study in Malaysia concluded that HM 25(OH)D was higher at delivery in women who used multivitamin supplement that contained 400 IU of vitamin D during pregnancy (Mohamed H J J, Rowan A, Fong B, Loy S L. Maternal serum and breast milk vitamin D levels: Findings from the Universiti Sains Malaysia pregnancy cohort study. PLoS One. 2014; 9(7):3-10). Similar to previous studies, increased HM vitamin D3 concentrations in the first 3 months of lactation with the NiPPeR intervention supplement can be thought to be mediated by improved maternal serum vitamin D concentrations during pregnancy in the intervention group. We observed that the NiPPeR intervention provided an additive source of vitamin D independent of that from sunlight exposure (Clark A, Mach N. Role of vitamin D in the hygiene hypothesis: The interplay between vitamin D, vitamin D receptors, gut microbiota, and immune response. Front Immunol. 2016; 7 (December):1-12), and the intervention effect was more evident in New Zealand site where seasonal variations in sunlight availability is greater than that in Singapore (Trenberth K E. What are the Seasons? Bull Am Meteorol Soc. 1983; 64(11):1276-82).

The beneficial effects of the NiPPeR intervention supplement on HM vitamin D concentrations could reflect a combined effect of dosage and duration of supplementation. The NiPPeR intervention supplement provided 400 IU of vitamin D₃ daily, which is the recommended intake for women (Department of Health. Dietary reference values: a guide. London: HMSO Publications Centre 1991; Dietetics Department NUH. Vitamins & Minerals Chart. National University Hospital. Singapore 2006; and National Health and Medical Research Council, Australian Government Department of Health and Ageing, New Zealand Ministry of Health. Nutrient Reference Values for Australia and New Zealand Including Recommended Dietary Intakes. Canberra: National Health and Medical Research Council 2006). Additionally, the average duration of supplementation from preconception to birth was 393.1 days in the intervention group, the total exposure of vitamin D3 being 157,240 IU. In comparison, Wagner et al. (Wagner C L, Hulsey T C, Fanning D, Ebeling M, Hollis B W. High-dose vitamin D3 supplementation in a cohort of breastfeeding mothers and their infants: a 6-month follow-up pilot study. Breastfeed Med. 2006; 1(2):59-70) observed a minimal increase in maternal serum 25(OH)D, vitamin D₃ and milk antirachitic activity with daily supplementation of 400 IU of vitamin D₃ for 6 months during lactation. Here, the total exposure of about 72,000 IU was lower than that achieved by the NiPPeR intervention supplement. Studies of maternal vitamin D supplementation in lactation with high dosage essentially focused on indirect treatment of the infant with increased HM vitamin D concentrations. Our study is distinctive from previous reports as regards window of effect whereby we show that supplementation with a lower dose from preconception and throughout pregnancy provided potential benefit to mothers throughout pregnancy as well as to offspring in early infancy.

No significant effects of the NiPPeR supplement were observed on HM B-vitamins, despite B₂ and B₆ only being present in the intervention drink. As B-vitamins are water soluble and there are no storage mechanisms in the body (Bellows L, Moore R, Anderson J, Young L. Water-Soluble Vitamins: B-Complex and Vitamin C. Vol. no. 9.312, Food and Nutrition Series. Health. 2012), it would not be unexpected that B-vitamins supplemented in preconception and pregnancy had no impact on HM concentrations during lactation. Other studies have shown an acute increase in HM B-vitamin concentrations with supplementation during lactation (Prentice A M, Roberts S B, Prentice A, Paul A A, Watkinson M, Watkinson A A, et al. Dietary supplementation of lactating Gambian women. I. Effect on breast-milk volume and quality. Hum Nutr Clin Nutr. 1983 January; 37(1):53-64; and Hamaker B, Kirksey A, Ekanayake A, Borschel M. Analysis of B-6 vitamers in human milk by reverse-phase liquid chromatography. Am J Clin Nutr [Internet]. 1985 Oct. 1; 42(4):650-5. Available from: https://doi.org/10.1093/ajcn/42.4.650) but very few studies have investigated such effects following supplementation during pregnancy.

In the New Zealand site, we observed an increasing trend of HM vitamin D concentrations from 1 week to 12 months of lactation, with a steady phase between 3 months to 6 months. Previously, Wagner et al. reported similar findings of rapid increases in milk antirachitic activity (sum of vitamin D₃ and 25(OH)D₃ concentrations) from 1 to 2 months, followed by a steady phase until 4 months, then continual increase until 7 months of lactation (Wagner C L, Hulsey T C, Fanning D, Ebeling M, Hollis B W. High-dose vitamin D3 supplementation in a cohort of breastfeeding mothers and their infants: a 6-month follow-up pilot study. Breastfeed Med. 2006; 1(2):59-70). However, this was only displayed in women who were supplemented a large daily dose of vitamin D (6,400 IU) during the study period while the 400 IU dose group showed little change. Other studies have reported inconsistent results of longitudinal changes of HM vitamin D concentrations. In Malaysian women, HM 25(OH)D decreased from delivery until 6 months then increased until 12 months of lactation (Mohamed H J J, Rowan A, Fong B, Loy S L. Maternal serum and breast milk vitamin D levels: Findings from the Universiti Sains Malaysia pregnancy cohort study. PLoS One. 2014; 9(7):3-10). In unsupplemented Japanese women, vitamin D₃ concentrations reached a maximum at 3 months then steadily decreased until 12 months (Sakurai T, Furukawa M, Asoh M, Kanno T. Fat-Soluble and Water-Soluble Vitamin Contents Breast Milk from Japanese Women of Nutritional Research Institute, Meiji Dairies Corporation, The vitamin contents of human milk from well-nourished women and the respective intakes of their exclusively b. 2005; 239-47). Oberhelman et al. reported a rapid decline of HM vitamin D3 during the first month of lactation, after reaching a peak at day 1 post-delivery (Oberhelman S S, Meekins M E, Fischer P R, Lee B R, Singh R J, Cha S S, Gardner B M, Pettifor J M, Croghan I T T. Status of Breastfed Infants: a Randomized Control Trial. J Clin Endocrinol Metab. 2013; 93(7):2693-701). However, due to the differences in study design, observational period and analytic methods, it is difficult to make direct comparisons between the current study and previous observations. The pattern was observed in both the intervention and the control group, evident not only in the intervention group, suggesting temporal changes in HM vitamin D throughout lactation was not affected by the NiPPeR intervention supplement. The mechanism behind increasing HM vitamin D concentrations with progressing lactation can be speculated. Studies that have assessed the relation between lactation and weight loss have reported greater weight loss among breastfeeding women compared to non-breastfeeding women (Butte N F, Hopkinson J M, Mehta N, Moon J K, Smith E O B. Adjustments in energy expenditure and substrate utilization during late pregnancy and lactation. Am J Clin Nutr. 1999; 69(2):299-307) and fat mobilisation appeared to increase after the first 3 months postpartum (Brewer M M, Bates M R, Vannoy L P. Postpartum changes in maternal weight and body fat depots in lactating vs nonlactating women. Am J Clin Nutr [Internet]. 1989 Feb. 1; 49(2):259-65. Available from: https://doi.org/i0.1093/ajcn/49.2.259; and Sadurskis A, Kabir N, Wager J, Forsum E. Energy metabolism, body composition, and milk production in healthy Swedish women during lactation. Am J Clin Nutr [Internet]. 1988 Jul. 1; 48(1):44-9. Available from: https://doi.org/10.1093/ajcn/48.1.44). While the main purpose of fat mobilization is thought to meet the energy demands of breastfeeding, vitamin D stored in fat (Abbas M A. Physiological functions of Vitamin D in adipose tissue. J Steroid Biochem Mol Biol. 2017; 165:369-81) may also be released into circulation in this process, thus increasing availability for transport into HM. Others have proposed that with increased outdoor activity as infants get older (Wagner C L, Hulsey T C, Fanning D, Ebeling M, Hollis B W. High-dose vitamin D3 supplementation in a cohort of breastfeeding mothers and their infants: a 6-month follow-up pilot study. Breastfeed Med. 2006; 1(2):59-70), mothers' sunlight exposure increases, allowing them to maintain optimal vitamin D status which may further lead to increase in HM vitamin D content.

Across the B-vitamins, varying patterns of change over lactation were observed. Vitamin B₁, B₂, B₃ all increased initially from 1 week to 6 weeks. Then, B₁ and B₃ decreased gradually until 12 months while B₂ concentrations remained fairly constant. Vitamin B₆ concentrations showed a similar pattern, increasing from 1 week to 3 months then decreasing until 12 months of lactation. In contrast, vitamin B₉ concentrations showed little change in the first 12 months of lactation.

Similar to our findings, Redeuil et al. observed an increase in HM vitamin B₁ concentrations in the first 6 weeks of lactation, followed by a plateau until 16 weeks (Redeuil K, Lévêques A, Oberson J M, Bénet S, Tissot E, Longet K, et al. Vitamins and carotenoids in human milk delivering preterm and term infants: Implications for preterm nutrient requirements and human milk fortification strategies. Clin Nutr. 2021; 40(1):222-8). Separately, free thiamine concentrations were previously found to increase over the course of lactation (Hampel D, Shahab-Ferdows S, Adair L S, Bentley M E, Flax V L, Jamieson D J, et al. Thiamin and riboflavin in human milk: Effects of lipid-based nutrient supplementation and stage of lactation on vitamer secretion and contributions to total vitamin content. PLoS One. 2016; 11(2):e0149479; Ford J E, Zechalko A, Murphy J, Brooke O G. Comparison of the B vitamin composition of milk from mothers of preterm and term babies. Arch Dis Child. 1983; 58(5):367-72; Ren X, Yang Z, Shao B, Yin S A, Yang X. B-vitamin levels in human milk among different lactation stages and areas in China. PLoS One. 2015; 10(7):1-12; and Xue Y, Redeuil K M, Gimenez E C, Vinyes-Pares G, Zhao A, He T, et al. Regional, socioeconomic, and dietary factors influencing B-vitamins in human milk of urban Chinese lactating women at different lactation stages. BMC Nutr. 2017; 3(1):1-11). Of note, Hampel et al. observed an increase in free and thiamine while TMP concentrations fell from 2 to 24 weeks postpartum. During this time, contribution of free thiamine and TMP to total thiamine increased from 7-11% to 26-27% and decreased from 88% to 71-72%, respectively (Hampel D, Shahab-Ferdows S, Adair L S, Bentley M E, Flax V L, Jamieson D J, et al. Thiamin and riboflavin in human milk: Effects of lipid-based nutrient supplementation and stage of lactation on vitamer secretion and contributions to total vitamin content. PLoS One. 2016; 11(2):e0149479), comparable to our observations.

Previous studies have reported that HM vitamin B₂ and FAD concentrations reach maximum at 6 weeks (Redeuil K, Lévêques A, Oberson J M, Bénet S, Tissot E, Longet K, et al. Vitamins and carotenoids in human milk delivering preterm and term infants: Implications for preterm nutrient requirements and human milk fortification strategies. Clin Nutr. 2021; 40(1):222-8) and at 8-14 days of lactation (Ren X, Yang Z, Shao B, Yin S A, Yang X. B-vitamin levels in human milk among different lactation stages and areas in China. PLoS One. 2015; 10(7):1-12), respectively. Others have observed that total HM riboflavin decreased from 2 to 24 weeks of lactation, attributable to a reduction in FAD (Hampel D, Shahab-Ferdows S, Adair L S, Bentley M E, Flax V L, Jamieson D J, et al. Thiamin and riboflavin in human milk: Effects of lipid-based nutrient supplementation and stage of lactation on vitamer secretion and contributions to total vitamin content. PLoS One. 2016; 11(2):e0149479), the major form of vitamin B₂. Beyond this time point, we observed that total HM vitamin B₂ concentrations remain fairly constant from 6 to 12 months of lactation.

HM vitamin B₃ concentrations have been reported to reach a peak between 1-3 weeks of lactation then gradually decrease over time (Redeuil K, Lévêques A, Oberson J M, Bénet S, Tissot E, Longet K, et al. Vitamins and carotenoids in human milk delivering preterm and term infants: Implications for preterm nutrient requirements and human milk fortification strategies. Clin Nutr. 2021; 40(1):222-8; and Ren X, Yang Z, Shao B, Yin S A, Yang X. B-vitamin levels in human milk among different lactation stages and areas in China. PLoS One. 2015; 10(7):1-12). From the current study, we were able to identify that this reduction in HM vitamin B₃ over time was driven by NMN, on average representing 81% of total vitamin B₃.

Similarly, HM vitamin B₆ concentrations reached a maximum at 3 months of lactation then remained fairly constant until 12 months. Redeuil et al. also reported similar findings of increased HM vitamin B₆ concentrations in the first 6 weeks then remaining stable until 16 weeks (Redeuil K, Lévêques A, Oberson J M, Bénet S, Tissot E, Longet K, et al. Vitamins and carotenoids in human milk delivering preterm and term infants: Implications for preterm nutrient requirements and human milk fortification strategies. Clin Nutr. 2021; 40(1):222-8). However, there is other evidence suggesting an increase in HM vitamin B6 over time. In a Chinese study, the maximum vitamin B6 concentration was reached in HM collected between 181-330 days (Ren X, Yang Z, Shao B, Yin S A, Yang X. B-vitamin levels in human milk among different lactation stages and areas in China. PLoS One. 2015; 10(7):1-12). In a study by Ford et al., the maximum was achieved in HM collected between 16-244 days (Ford J E, Zechalko A, Murphy J, Brooke O G. Comparison of the B vitamin composition of milk from mothers of preterm and term babies. Arch Dis Child. 1983; 58(5):367-72). These conflicting observations may be due to the differences in the B₆-vitamer analysed or the analytic methods used. Further, these other studies have used a wide HM sampling period across lactation whereas the current study assessed HM collected within tight sampling windows.

Previously, HM vitamin B₉ concentrations have been reported to increase progressively over the first several weeks of lactation (Redeuil K, Lévêques A, Oberson J M, Bénet S, Tissot E, Longet K, et al. Vitamins and carotenoids in human milk delivering preterm and term infants: Implications for preterm nutrient requirements and human milk fortification strategies. Clin Nutr. 2021; 40(1):222-8; and Ford J E, Zechalko A, Murphy J, Brooke O G. Comparison of the B vitamin composition of milk from mothers of preterm and term babies. Arch Dis Child. 1983; 58(5):367-72). Likewise, we observed that HM vitamin B₉ concentrations peaked at 3 months and beyond this time point, gradually decreased until 12 months. On the other hand, 5MeTHF, contributing to 710% of total HM vitamin B₉ on average, did not change greatly over the first year of lactation.

### Strengths and Limitations

Using a gold-standard, randomised, controlled trial, this study investigated the impact of a nutritional supplementation from preconception and during pregnancy on HM vitamin composition during lactation. Through an international, multicentral study design, we examined HM vitamin composition in a large population of diverse ethnic groups. To overcome potential variations at each study site, Singapore and New Zealand sites have used a standardized HM sample collection, processing and vitamin quantification methods. During this process, whole milk samples were collected in order to avoid potential diurnal variations in HM composition. In addition, we have tightly controlled the visit window, each HM collection time point being a distinctive stage of lactation. This allowed to describe the changes in HM vitamin concentrations between the different stages over the course of lactation. However, due to logistical constraints, longitudinal samples could not be collected from all participants. To address the imbalance of sample number at each time point, a repeated measures design was used for statistical analyses, along with sensitivity analyses in a subgroup of mother with consecutive samples collected. In this study, infant blood was not collected, limiting the further assessment of the relationship between vitamin concentrations in HM and in infant circulation. However, collecting infant blood samples is a challenging task, requiring strong justification and ethical considerations.

### CONCLUSIONS

Nutritional supplement from preconception through pregnancy with lower dose of vit D achieved higher levels of vitamin D₃ concentrations during the first 3 months of lactation. In the future, ongoing evaluation of infants from this cohort is required to understand the implications and impact of HM vitamin compositions on infant outcomes such as growth, bone health, immunity, adiposity and diabetes during later childhood.

1. A method for increasing at least one micronutrient level in breastmilk of a subject, the method comprising administering to the subject a nutritional composition comprising:
at least one of vitamin D or zinc.

2. The method of claim 1, wherein the nutritional composition is administered to the subject from before pregnancy of the subject.

3. The method of claim 2, wherein the nutritional composition is administered to the subject until the subject gives birth.

4. The method of claim 1, wherein the nutritional composition further comprises at least one of:
myo-inositol, probiotics, vitamin B2, vitamin B6, or vitamin B12.

5. The method of claim 1, wherein the nutritional composition further comprises at least one of:
folic acid, Fe, Ca, I and beta-carotene.

6. The method of claim 1, wherein the nutritional composition comprises vitamin D and zinc.

7. The method of claim 1, wherein the increasing of the at least one micronutrient level is sustained for at least 12 months after the subject gives birth.

8. The method of claim 1, wherein the nutritional composition is administered in a daily dose comprising
between about 1.5 g and about 100 g vitamin D; and/or
between about 1.1 mg and about 40 mg zinc.

9. The method of claim 1, wherein the nutritional composition is administered in a daily dose comprising
about 10 g vitamin D; and/or
about 10 mg zinc.

10. The method of claim 1, wherein the nutritional composition is administered in a daily dose comprising
between about 0.2 g and about 11 g myo-inositol;
between about 10′ and about 10¹² colony forming units (cfu) probiotics comprising B. lactis and LPR;
between about 0.14 mg and about 14 mg vitamin B2;
between about 0.19 mg and about 19 mg vitamin B6; and/or
between about 0.26 μg and about 26 μg vitamin B12.

11. The method of claim 1, wherein the nutritional composition is administered in a daily dose comprising
in a daily dose comprising about 4 g myo-inositol;
about 10⁹ cfu probiotics comprising B. lactis and LPR;
about 1.8 mg a daily dose of vitamin B2;
about 2.6 mg vitamin B6; and/or
about 5.2 μg vitamin B12.

12. The method of claim 1, wherein the nutritional composition is administered in a daily dose comprising
between about 60 μg and about 1000 μg folic acid;
between about 2.7 mg and about 45 mg iron;
between about 10 mg and about 2500 mg calcium;
between about 22 μg and about 1100 μg iodine; and/or
between about 77 μg and about 3000 μg beta-carotene.

13. The method of claim 1, wherein the nutritional composition is administered in a daily dose comprising
about 400 μg folic acid;
about 12 mg iron;
about 150 mg calcium;
about 150 μg iodine; and/or
about 400 μg beta-carotene.

14. The method of claim 1, wherein the subject is a female mammal who is pregnant or trying to become pregnant.

15. The method of claim 1, wherein the subject is selected from the group consisting of a cat, a dog and a human.

16. The method of claim 1, wherein the vitamin D is vitamin D3.

17. The method of claim 2, wherein the nutritional composition is administered to the subject when the subject is considering to become pregnant.
